SEQUENCE LISTING (187 sequences)

SEQ ID NO: 1 - DNA, 772 bp, Staphylococcus aureus
  misc_feature 4, 6: n = A,T,C or G
tcangngggatcaaacggcctgcacaaggacgtcttacaacgcagtaactacgcactatcattcagcaaaatgacatttccacatcaaatgatgcgggttgtgttaattgagcaagtgtatagagcgtttaagattatgcgcggagaagcgtatcacaaatgatgcggtttttttaacctctttacgtatgaggtttatgagaattgccgttatgttttgcgagagttatcaatctttttgatagtaagaaagtacatagaaactaaaagagtatttttatctacaatagcatttataatttattctattattgtatacttattttaattattagtatcattgctgagatgttacttgatattctatgtctattttttaggaaattctatactattaaaattatggtattttatacgcaataaaggactaatctattttatacagattagtcctttattgtagtctttaaaaactagttactcattaatattttttagtacaatttcagcaacctcacttactattttgtcattaggtttaccatcttttctatctttatttgtaaatatcaccagaattataggtttatcttgtccatctggataaacaaagcgaacatcgtttcttgaaccgtatgttagtgcttgaccgctcttatccataactttaaagtttgaaggtgcaccatccttaattaatgtatcgccacttttatttttgaacattagattaagtaagaaatctttgtttgctttgctaagatctccatca

SEQ ID NO: 2 - DNA, 21 bp, Artificial Sequence
  Synthesized Oligonucleotide
cggcaattctcataaacctca

SEQ ID NO: 3 - DNA, 19 bp, Artificial Sequence
  Synthesized Oligonucleotide
ggatcaaacggcctgcaca

SEQ ID NO: 4 - DNA, 24 bp, Artificial Sequence
  Synthesized Oligonucleotide
tagttactgcgttgtaagacgtcc

SEQ ID NO: 5 - DNA, 3050 bp, Staphylococcus aureus
  Synthesized Oligonucleotide
tcgtgccattgatgcagagggacatacattagatatttggttgcgtaagcaacgagataatcattcagcatatgcgtttatcaaacgtctcattaaacaatttggtaaacctcaaaaggtaattacagatcaggcaccttcaacgaaggtagcaatggctaaagtaattaaagcttttaaacttaaacctgactgtcattgtacatcgaaatatctgaataacctcattgagcaagatcaccgtcatattaaagtaagaaagacaaggtatcaaagtatcaatacagcaaagaatactttaaaaggtattgaatgtatttacgctctatataaaaagaaccgcaggtctcttcagatctacggattttcgccatgccacgaaattagcatcatgctagcaagttaagcgaacactgacatgataaattagtggttagctatatttttttactttgcaacagaaccgaaaataatctcttcaatttatttttatatgaatcctgtgactcaatgattgtaatatctaaagatttcagttcatcatagacaatgttcttttcaacattttttatagcaaattgattaaataaattctctaatttctcccgtttgatttcactaccatagattatattatcattgatatagtcaatgaataatgacaaattatcactcataacagtcccaacccctttattttgatagactaattatcttcatcattgtaaaacaaattacaccctttaaatttaactcaacttaaatatcgacaaattaaaaaacaataaaattacttgaatattattcataatatattaacaactttattatactgctctttatatataaaatcattaataattaaacaagccttaaaatatttaacttttttgtgattattacacattatcttatctgctctttatcaccataaaaatagaaaaaacaagattcctaaagaatataggaatcttgtttcagactgtggacaaactgattttttatcagttagcttatttagaaagttttatttaaattacagtttctatttttattagatcacaattttattttagctcttgttcaagtaatcatttttcgccaaaaactttatactgaatagcttctacattaaatactttgtcaatgagatcatctacatctttaaattcagaataatttgcatatggatctataaaataaaattgtggttctttaccggaaacattaaatattcttaatattaaatatttctgcttatattctttcatagcaaacatttcatttagcgacataaaaaatggttcctcaatactagaagatgtagatgttttaatttcaataaatttttctacagctttatctgtatttgttggatcaaaagctactaaatcatagccatgaccgtgttgagagcctggattatcatttaaaatattcctaaactgttctttcttatcttcgtctattttattatcaattagctcattaaagtaatttagcgctaatttttctccaactttaccggttaatttattctctttatttgatttttcaatttctgaatcatttttagtagtctttgatacaccttttttatattttggaattattcctttaggtgcttccacttccttgagtgtcttatctttttgtgctgttctaatttcttcaatttcgctgtcttcctgtatttcgtctatgctattgaccaagctatcataggatgtttttgtaacttttgaagctaattcattaaatagttctaaaaatttctttaaatcctctagcatatcttcttctgtgaatccttcattcaaatcataatatttgaatcttattgatccatgagaatatcctgatggataatcattttttaaatcataagatgaatctttattttctgcgtaataaaatcttccagtattaaattcatttgatgtaatatatttattgagttcggaagataaagttaatgctctttgttttgcagcatttttatcccgcggaaacatatcacttatctttgaccatccttgattcaaagataagtatatgccttctccttccggatgaaaaagatataccaaataatatccatcctttgtttcttttgttatattctcatcatatattgaaatccaaggaactttactatagttcccagtagcaaccttccctacaactgaatatttatcttcttttatatgcacttttaactgcttgggtaacttatcatggactaaagttttatatagatcacctttatcccaatcagattttttaactacattattggtacgtttctctttaattaatttaaggacctgcataaagttgtctatcatttgaaattccctcctattataaaatatattatgtctcattttcttcaatatgtacttatttatattttaccgtaatttactatatttagttgcagaaagaattttctcaaagctagaactttgcttcactataagtattcagtataaagaatatttcgctattatttacttgaaatgaaagactgcggaggctaactatgtcaaaaatcatgaacctcattacttatgataagcttctcctcgcataatcttaaatgctctgtacacttgttcaattaacacaacccgcatcatttgatgtgggaatgtcattttgctgaatgatagtgcgtagttactgcgttgtaagacgtccttgtgcaggccgtttgatccgccaatgacgaaaacaaagtcgctttgcccttgggtcatgcgttggttcaattcttgggccaatccttcggaagatagcatctttccttgtatttctaatgtaatgactgtggattgtggtttgattttggctagtattcgttggccttctttttcttttacttgctcaatttctttgtcactcatattttctggtgctttttcgtctggaacttctatgatgtctatcttggtgtatgggcctaaacgtttttcatattctgctatggcttgcttccaatatttctcttttagtttccctacagctaaaatggtgattttcatgtc

SEQ ID NO: 6 - DNA, 3050 bp, Staphylococcus aureus
acctcattgagcaagatcaccgtcatattaaagtaagaaagacaaggtatcaaagtatcaatacagcaaagaatactttaaaaggtattgaatgtatttacgctctatataaaaagaaccgcaggtctcttcagatctacggattttcgccatgccacgaaattagcatcatgctagcaagttaagcgaacactgacatgataaattagtggttagctatatttttttactttgcaacagaaccgaaaataatctcttcaatttatttttatatgaatcctgtgactcaatgattgtaatatctaaagatttcagttcatcatagacaatgttcttttcaacattttttatagcaaattgattaaataaattctctaatttctcccgtttgatttcactaccatagattatattatcattgatatagtcaatgaataatgacaaattatcactcataacagtcccaacccctttcttttgatagactaattatcttcatcattgtaaaacaaattacaccctttaaatttaactcaacttaaatatcgacaaattaaaaaacaataaaattacttgaatattattcataatatattaacaactttattatactgctctttatatataaaatcattaataattaaacaagccttaaaatatttaacttttttgtgattattacacattatcttatctgctctttatcaccataaaaatagaaaaaacaagattcctaaagaatataggaatcttgtttcagactgtggacaaactgattttttatcagttagcttatttagaaagttttatttaaattacagtttctatttttattagatcacaattttattttagctcttgttcaagtaatcatttttcgccaaaaactttatactgaatagcttctacattaaatactttgtcaatgagatcatctacatctttaaattcagaataatttgcatatggatctataaaataaaattgtggttctttaccggaaacattaaatattcttaatattaaatatttctgcttatattctttcatagcaaacatttcatttagcgacataaaaaatggttcctcaatactagaagatgtagatgttttaatttcaataaatttttctacagctttatctgtatttgttggatcaaaagctactaaatcatagccatgaccgtgttgagagcctggattatcatttaaaatattcctaaactgttctttcttatcttcgtctattttattatcaattagctcattaaagtaatttagcgctaatttttctccaactttaccggttaatttattctctttatttgatttttcaatttctgaatcatttttagtagtctttgatacaccttttttatattttggaattattcctttaggtgcttccacttccttgagtgtcttatctttttgtgctgttctaatttcttcaatttcgctgtcttcctgtatttcgtctatgctattgaccaagctatcataggatgtttttgtaacttttgaagctaattcattaaatagttctaaaaatttctttaaatcctctagcatatcttcttctgtgaatccttcattcaaatcataatatttgaatcttattgatccatgagaatatcctgatggataatcattttttaaatcataagatgaatctttattttctgcgtaataaaatcttccagtattaaattcatttgatgtaatatatttattgagttcggaagataaagttaatgctctttgttttgcagcatttttatcccgcggaaacatatcacttatctttgaccatccttgattcaaagataagtatatgccttctccttccggatgaaaaagatataccaaataatatccatcctttgtttcttttgttatattctcatcatatattgaaatccaaggaactttactatagttcccagtagcaaccttccctacaactgaatatttatcttcttttatatgcacttttaactgcttgggtaacttatcatggactaaagttttatatagatcacctttatcccaatcagattttttaactacattattggtacgtttctctttaattaatttaaggacctgcataaagttgtctatcatttgaaattccctcctattataaaatatattatgtctcattttcttcaatatgtacttatttatattttaccgtaatttactatatttagttgcagaaagaattttctcaaagctagaactttgcttcactataagtattcagtataaagaatatttcgctattatttacttgaaatgaaagactgcggaggctaactatgtcaaaaatcatgaacctcattacttatgataagcttcttaaaaacataacagcaattcacataaacctcatatgttctgatacattcaaaatccctttatgaagcggctgaaaaaaccgcatcatttatgatatgcttctccacgcataatcttaaatgctctatacacttgctcaattaacacaacccgcatcatttgatgtgggaatgtcattttgctgaatgatagtgcgtagttactgcgttgtaagacgtccttgtgcaggccgtttgatccgccaatgacgaatacaaagtcgctttgcccttgggtcatgcgttggttcaattcttgggccaatccttcggaagatagcatctttccttgtatttctaatgtaatgactgtggattgtggtttaattttggctagtattcgttggccttctttttcttttacttgctcaatttctttgtcgctcatattttctggtgctttttcgtctggaacttctatgatgtctatcttggtgtatgggcctaaacgtttttcatattctgctatggcttgcttccaatatttctcttttagtttccctacagctaaaatggtgattttcatgtcgtttggtcctccaaattgttatcaactttccagttatccacaagttattaacttgttcacactgttccctcttattataccaatattttttgcagtttttgatattttcctgacattta

SEQ ID NO: 7 - DNA, 1256 bp, Staphylococcus aureus
ttcagaaaaatgattaatgtgtttcaataaaatctctccttctttgtgaacatattcatttttatactaattaatataatttccaaaaaagtttctgtttaaaagtgaaaaatattatttaccgtttgacttaaatcttcaatatataggtgtttatatgtatcattttgcgccaatttgaataaacgggaatcaagtctgtttctgagtttatttcaactttcttatagtaaacattgtcttaatatgatgaacttcaataaaactttccctatgccccataaaattttctcaaaatcaaaaataacataccttacaacttttaccgtcgatatcaattgctcttttcttaatttaggattgctttcaaattttgtactataacgtgaaactacttttccttctttataattaaaatttactaattcacaatcatttttacttccatttacaaaaacatccactgtttctaacacaaaatctaataaacttccttttattaatcgtaggcattgtatatttcctttcattctttcttgattccattagtttaaatttaaaatttcatccatcaatttcttaatttaattgtagttccataatcaatataatttgtacagttattatatattctagatcatcaatagttgaaaaatggtttattaaacactctataaacatcgtatgatattgcaaggtataatccaatatttcatatatgtaattcctccacatctcattaaatttttaaattatacacaacctaatttttagttttatttatgatacgcttctccacgcataatcttaaatgctctgtacacttgttcaattaacacaacccgcatcatttgatgtgggaatgtcattttgctgaatgatagtgcgtagttactgcgttgtaagacgtccttgtgcaggccgtttgatccgccaatgacgaatacaaagtcgctttgcccttgggtcatgcgttggttcaattcttgggccaatccttcggaagatagcatctttccttgtatttctaatgtaatgactgttgattgtggtttgattttggctagtattcgttggccttctttttcttttacttgctcaatttctttgtcgctcatattttctggtgctttttcgtctggaacttctatgatgtctatcttggtgtatgggcctaaacgtttttcatattctgctatggcttgcttccaatatttctcttttagtttccctacagctaaaatggtgattttcat

SEQ ID NO: 8 - DNA, 2122 bp, Staphylococcus aureus
ggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcgtaccacaaatgatgcggttttttatccagttttttgtttaatgaacaaggtaaattacgagataatatttgaagaaaacaataaagtagagatggatttccatatcctctttagtagcggtttttatctgtaaggtttattaataattaaataaataggcgggatagttatatatagcttattaatgaaagaatatgattattaatttagtattatattttaatattaaaaagaagatatgaaataattattcataccttccaccttacaataattagttttcaatcgaatattaagattattagtagtcttaaaagttaagacttccttatattaatgacctaatttattatttgcctcatgaattatctttttatttctttgatatgtcccaaaccacatcgtgatatacactacaataaatattatgatgaaactaataatattctcaaagttcagatggaaccaacctgctagaatagcgagtgggaagaataggattatcatcaatataaagtgaactacagtctgttttgttatactccaatcggtatctgtaaatatcaaattaccataagtaaacaaaattccaatcaatgcccatagtgctacacatattagcataataaccgcttcattaaagttttcataataaattttacccataaaagaatctggatatagtggtacatatttatcccttgaaaaaaataagtgaagtaatgacagaaatcataagaccagtgaacgcacctttttgaacagcgtggaataattttttcatagtgagatggaccattccatttgtttctaacttcaagtgatcaatgtaatttagattgataatttctgattttgaaatacgcacgaatattgaaccgacaagctcttcaatttggtaaagtcgctgataaagttttaaagctttattattcattgttatcgcatacctgtttatcttctactatgaactgtgcaatttgttctagatcaattgggtaaacatgatggttctgttgcaaagtaaaaaaatatagctaaccactaatttatcatgtcagtgttcgcttaacttgctagcatgatgctaatttcgtggcatggcgaaaatccgtagatctgatgagacctgcggttctttttatatagagcgtaaatacattcaataccttttaaagtattctttgctgtattgatactttgataccttgtctttcttactttaatatgacggtgatcttgctcaatgaggttattcagatatttcgatgtacaatgacagtcaggtttaagtttaaaagctttaattactttagccattgctaccttcgttgaaggtgcctgatctgtaattaccttttgaggtttaccaaattgtttaatgagacgtttgataaacgcatatgctgaatgattatctcgttgcttacgcaaccaaatatctaatgtatgtccctctgcatcaatggcacgatataaatagctccattttccttttattttgatgtacgtctcatcaatacgccatttgtaataagcttttttatgctttttcttccaaatttgatacaaaattggggcatattcttgaacccaacggtagaccgttgaatgatgaacgtttacaccacgttcccttaatatttcagatatatcacgataactcaatgtatatctta

SEQ ID NO: 9 - DNA, 1696 bp, Staphylococcus aureus
aaagagaaatattggaagcaagccatagcagaatatgaaaaacgtttaggcccatacaccaagatagacatcatagaagttccagacgaaaaagcaccagaaaatatgagtgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgttttcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgaggagaagcatatcataaatgatgcggttatttcagccgtaattttataatataaagcagagtttattaaattttaatgattactttttattaagaattaattctagttgatatattataatgtgaaacacaaaataataatttgtaattgttagtttataggcatctgtatttggaattttttgtagactatttaaaaaatagtgtatataagtattgagttcatgtattaactgtcttttttcatcgttcatcaagtataaggatgtagagatttgttggataatttcttcggatgtttttaaaattatcattaaattagatggtatctgatcttgagttttgtttttagtgtatgtatattttaaaaaatttttgattgttgttatttgactctcttttaatttgacaccctcatcaataaatgtgttaaatatatcttcatttgtacttaaatcatcaaaatttgccaacaaatatttgaacgtctctaaatcattatgtttgagttccgttttgctattccataattccaaaccatttggtagaaagcccaagctgtgattttgatctccccatatagctgaatttaaatcagtgagttgattaattttttcaacacagaaatgtaattttggaatgaggaatcgaagttgttcttctacttgctgtacttttcttttgttttcaataaaatttctacaccatactgttatcaaaccgccaattattgtgcacaatcctccaatgattgtagataaaattgacaatatattacacacctttcttagaggtttattaacatctatttttgaatttaaaattattactttggtagcgttataacctatttaacagattagagaaaaattgaatgatcgattgaagaatttccaaaataccgtcccatatgcgttgaaggagatttctattttcttctgtattcaaatctttggctttatcctttgctttattcaataaatcatctgagtttttttcaatattttttaatacatctttggcattttgtttaaatactttaggatcggaagttagggcattagagtttgccacattaatcatattattattaatcatttgaatttgattatctgataatatctctgataacctacgctcatcgaggactttattaacagtgtcttcaacttgttgttgtgtgatttgtttatcttgattttgtttaatatctgcaagttgttctttaatatctgctatagaagcatttaaagcttcatctgaatacccat

SEQ ID NO: 10 - DNA, 991 bp, Staphylococcus aureus
accattttagctgtagggaaactaaaagagaaatactggaagcaagccatagcagaatatgaaaaacgtttaggcccatacaccaagatagacatcatagaagttccagacgaaaaagcaccagaaaatatgaactacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgaggagaagcgtatcataagtgatggtaaaaaatatgagtaagtagatgaagagtgaaaatcagattaattaataataatgtatcaaatttaaataaaggggtttttaagtatgaatttaagaggtcatgaaaatagacttaaatttcatgcgaaatatgatgtgacacctatatcacatttaaaattattagaaggtcaaaagaaagacggtgaaggcggcatactgacagatagctattactgtttttcatacagcttaaaaggtaattctaaaaaagttttaggtacgtttaattgtggttatcatattgctgaagatttactaaaattatcaaatcaagataaattacctttatttaacccgtttaaagtaattaatgaaggtaatcaattgcagggcgtaacgaataaaggtaatttaaatattaataggcaaagaaaacagtataatgaagtggctttacagctttcaaatgctattaatttaatcataatttgttatgaggataatattaaagaaccactttcaacgataaaatac

SEQ ID NO: 11 - DNA, 1282 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaagcgtaccacaaataaaactaaaaaatatgagaaaattattaaattagctcaaatctttgaagaataaaaagtgaatattaagtttgataatttaggtacaagtaaagattaagaatttccattatttaatacatggtgtgtaaatcgacttctttttgtattagatgtttgcagtaagcgatgtaaagaagatgctaataaatatgtgaggaatgattacgatactagataagcggctaatgaaattttttaaagtacatatatagacatatttttcatttagtaaaattttgaatttcactttgctaagactagtgtctagaaatttataatgatttattaacacctatttgaaacttaagtataataaatgattcggattttatttttaataaagacaaacttgaacgtagcaaagtagtttttatgataaataataagttttaataatgtgacgcttttatataagcacattattatgaacaatgtgaattgagcatctacaattacattaataaatatataaatgatgatttaaattcacatatatttataatacacatactatatgaaagttttgattatccgaataaatgctaaaattaataaaataattaaaggaatcatacttattatacgtatacgtttagctactgaactactggattcatttggagattctagtagttctttttcaatctctaaatctaaatcagttttgtaataaccattaattcctaatctttcatctagctctgtacttttttcatcatttttatctttgttgatatgttccattttctcgcctctttttaatcaagtagaa

SEQ ID NO: 12 - DNA, 1530 bp, Staphylococcus aureus
ttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaagcatatcataaatgatgcggttttttcagccgcttcataaaggggggtgatcatatcggaacgtatgaggtttatgagaattgctgctatgtttttatgaagcgtatcataaatgatgcagtttttgataattttttctttatcagagattttactaaaaatcccctcaaagtttgtttttttcaacttcaactttgaagggaataaataaggaacttatttatatttatcctttatctcattaatatctatttttttattaataatattataaatattaaattctttagaaaagtcactatcactcttattcttcatactaaacgttattaatctaataatatcagctactatttctttaaattctattgcatcttcttttttataagtagcgcctgtatgaacaattttatttctcataccatagtaatctttcatatatttttttacacaatttttaatttcattagaattatccaaatctagattatcaattgtctttaataaatgatcattaacaacattagcatacccacatccaagcttcttttttatctcttcatcacttaaattttcatctaatttataatatctttctaaaaaatttgtgataaaaacttctaatgcagtctgaatttgtacaattgctaaattatagtcagatttataaaaagaacgttcaccttttctcatagccaaaacataaatattgctaggatgattattgaaaatattataattttttttaatatttaataaatcactttttttgatagatgaatactgatcttcttctatctttccaggcatgtcaatcatgaaaatactcatctcttttatatttccatctatagtatatattatataatatggaatacttaatatatcccctaatgatagctggtatatattatgatactgatatttaacgctaataattttaataagattatttagacaattaaattgcttattaaaaattttcgttagactattacttttctttgattccctagaagtagaatttgatttcaattttttaaactgattgtgcttgattattgaagttatttcaacata

SEQ ID NO: 13 - DNA, 1256 bp, Staphylococcus aureus
gctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcatatcataaatgatgcggttttttcagccgcttcataaagggattttgaatgtatcagaacatatgaggtttatgtgaattgctgttatgtttttaagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatctaagttaattaatagaaaccagaaatatttcagtgtgagatctgctggaacaaaagtgaaaaatatttctaaaggacatgta

SEQ ID NO: 14 - DNA, 748 bp, Staphylococcus aureus
atcgtttaacgtgtcacatgatgcgatagatccgcaattttatattttccataataactataagaagtttacgattttaacagatacgggttacgtgtctgatcgtatgaaaggtatgatacgtggcagcgatgcatttatttttgagagtaatcatgacgtcgatatgttgagaatgtgtcgttatccatggaagacgaaacaacgcattttaggcgatatgggtcatgtatctaatgaggatgcgggtcatgcgatgacagacgtgattacaggtaacacgaaacgtatttacttatcgcatttatcacaagataataatatgaaagatttggcgcgtatgagtgttggccaagtattgaacgaacacgatattgatacggaaaaagaagtattgctatgtgatacggataaagctattccaacaccaatatatacaatataaatgagagtcatccgataaagttccgcactgctgtgaaacgactttatcgggtgcttttttatgttgttggtgggaaatggctgttgttgagttgaatcggattgattgaaatgtgtaaaataattcgatattaaatgtaatttataaataatttacataaaatcaaacattttaatataaggattatgataatatattggtgtatgacagttaatggagggaacgaaatgaaagctttattacttaaaacaagtgtatggctcgttttgctttttagtgtgatgggattatggcatgtctcga

SEQ ID NO: 15 - DNA, 1100 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcgtatcacaaataaaactaaaaaataagttgtatataacttattttgaaattggttaagtatataatatctccaataaaatgtagttaacttacgataatgctgaactatagctttgtaaactaaaatgtaaataattacaatcaaattgcaacaatatagttcaagaatgctacaatttgaggacagattgatagcattaatccctttaaaatgaagctaggagataacttacattatgattagtaaacaaataaaggatttacgaaagcaacataattatactcaagaagagctagctgaaaaattaaatacttcaagacaaacaatttctaaatgggaacaaggtatttcagaaccagacttaattatgcttatgcaattgtcacaattattttctgttagtacagactatctcattacaggaagtgacaatattattaaaaaagataataaaagctattatgaaatgaatttttgggcatttatgtctgaaaaatggtgggtaattattattatagtaatcataatttgtggaacaataggacaaattttttcaaactaatgtaagtatctctcaaatattttgggaggttttattatgaaaatcaaaaaattattaaagacattattaattattttat

SEQ ID NO: 16 - DNA, 1109 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcgtatcacaaataaaactaaaaaataagttgtatataacttattttgaaattggttaagtatatagtatctccaataaaatgtagttaacttacgataatgctgaactatagctttgtaaactaaaatgtaaataattacaatcaaattgcaacaatatagttcaagaatgctacaatttgaggacagattgatagcattaatccctttaaaatgaagctaggagataacttacattatgattagtaaacaaataaaggatttacgaaagcaacataattatactcaagaagagctagctgaaaaattaaatacttcaagacaaacaatttctaaatgggaacaaggtatttcagaaccagacttaattatgcttatgcaattgtcacaattattttctgttagtacagactatctcattacaggaagtgacaatattattaaaaaagataataaaagctattatgaaatgaatttttgggcatttatgtctgaaaaatggtgggtaattattattatagtaatcataatttgtggaacaataggacaaattttttcaaactaatgtaagtatctctcaaatattttgggaggttttattatgaaaatcaaaaaattattaaagacattattaattattttattatgttttg

SEQ ID NO: 17 - DNA, 1159 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcgtatcacaaataaaactaaaaaataagttgtatataacttattttgaaattggttaagtatatagtatctccaataaaatgtagttaacttacgataatgctgaactatagctttgtaaactaaaatgtaaataattacaatcaaattgcaacaatatagttcaagaatgctacaatttgaggacagattgatagcattaatccctttaaaatgaagctaggagataacttacattatgattagtaaacaaataaaggatttacgaaagcaacataattatactcaagaagagctagctgaaaaattaaatacttcaagacaaacaatttctaaatgggaacaaggtatttcagaaccagacttaattatgcttatgcaattgtcacaattattttctgttagtacagactatctcattacaggaagtgacaatattattaaaaaagataataaaagctattatgaaatgaatttttgggcatttatgtctgaaaaatggtgggtaattattattatagtaatcataatttgtggaacaataggacaaattttttcaaactaatgtaagtatctctcaaatattttgggaggttttattatgaaaatcaaaaaattattaaagacattattaattattttattatgttttgtattgtctgttattgtgcaaaatatttcaatgctatggcatattgtgagc

SEQ ID NO: 18 - DNA, 1125 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaggcgtatcataaataaaactaaaaaacggattgtgtataatatattttaaatataaaaaggattgattttatgttaaataaattagaaaatgttagttataaatcattcgataattacactagtgaagatgatttgactaaagtaaatatattttttggaagaaatgggagtggaaaaagctcattaagtgaatggttaagaagactagataatgaaaaaagtgttatctttaatactggttacttaaaaaataatattgaagaagttgaagaaatagatggtgtgaatttggttattggagaagaatctataaatcatagtgaccaaattaagcatttaaatagcgctataaatagtttagaaaattttattactcggaaaaatagtgaacttaagcattcaaaagaaagaatttacaataaaatgaatatcagactaaatgaagctagagaaagatttgaaataggtagtaatgtggttaagcagaagaggaatgctgacaaagatccagttaatgctttttatagttggaagaaaaatgctaacgatataattcaagagatgactattgaatctttagatgaattagaagaaagaataacaagaaaagaagtcttattaaataatataaaaacaccaattttagcttttgattataatgattttagt

SEQ ID NO: 19 - DNA, 4149 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatctaagttaattaatagaaaccagaaatatttcagtgtgagatctgctggaacaaaagtgaaaaatatttctaaaggacatgtagaaaactttaattttttatctcctaattacactgaacaacaaaaaataggtaatttcttcagcaaactcgaccgccagattgagttagaagaagagaaacttgaactcttagagcaacaaaagcgtggatatattcagaagattttttctcaagatttaagatttaaagatgaaaatggaaacagttatcctgattggtctattaaaaagattgaagatatttctaaagttaataaagggtttactccaaatacaaaaaatgataaatactgggatgaattaaatgaaaattggttatctatagcaggtatgacacagaaatatttgtataaaggaaataaaggaattactgaaaaaggtgcatcaaagcatgtaaaagtagataaagatactctaataatgagttttaaattgactttaggtaagttagctatagtaaaagagcctatctatacaaatgaagctatatgccatttcgtatggaaagaaagtaatgttaatactgagtatatgtactactatttaaattctataaatataagtacttttggtgcacaggcagttaaaggagtaacattaaataacgatgcaattaatagtattatagtaaagttaccagtgatacaagaacaaaataaaatagcatactttttcaataaattagataaattaattgaaaaacaatcttctaaagtagaattattaaaacaacgcaaacaaggatttttacagaaaatgtttgtttaattcttataaagttctattatgtaaaatattaaatagagataacattatgaaagcgagcccaagacataaagtttttgaataaataaaaaagataatttctatcaaattaatatagaaattgtcttttttataaattttttgattatttttagctgattgagctgttacttttcttataataagtgctattagcacaaatcctagttctcttttggctttgtttattcctcttacggacattcgagtgaaacccattttaattttattagaagtaatttaggtttgaacccacctaaataaatatatgagttatttttttatgctacaaaatatattcagatttcaataatgacataaaataggcatctttatatttacctttagtgtagaattgctctttgagtaatccttctgttttaaatccttgtgactcgtatatatgcacagcttttttgttatctgtatcaacatatagataaattttgtgcatgtttaatatatcgaatgcataatttatcgctttttcgaatgcgaattttgcataacctttaccactgaactcaggtttaataattatttgtatttcacaattacgatggatgtaattaatttctactaattcaacaatacctacgacttgattttcatcttcaacaataaaacgtctctctgattcatctaataaatgcttatcaaataaatattgaagttccgttaaggattcatatggttcttcaaaccaataagacataatagaatattcattatttaattcatgaacaaaaagtaaatcactatactctaatgctcttagtttcataattccactcccaaaattttctcatatatttgcattataaatataaataacgaataagtcatcattcactgtgaatactctattttaacaattcaccacatactaattctcattttcttgttattctcgatttattactcttactatgaaacctataaaattctcacatttgtttgtattaagaataaatacgtcgatagtaacaataaaaaaataaataataaagcatccctcaccgtaaaagtgaaggatgctctagttttattgaaatatacatttcattttgttaaataattattaataatattttgaaaatcattattacgtgaaatcttcatagattttatcaagtatttctttgccttcaattgctgtgaagtgatgtaccaatctatttttacaatcatatgtaattttgtgacgctaggtaattagtaattgttcgtcagtctgattgtatagtatcaagtttcatagataatactctttgattttaatgtccactttgacgtgctttaagattgagtatatacataatgtcattgtggaatgttaaaaatcctacaaatgtttattcatctgcaggatttttaaatctccaagaataaaaatcatcataggacaactggattattgtttggataaataacgtaaacaataattaggtactattatttatttttgtttattctttttcctaacaaaataaagaaaagaataaacgcaattgttaaaaatatgtgtcctaaaccagcaataccagcaatagcaggacttacacttagatctttaatggtagaaataccgttcacgaattgcattgccacagtaacaagcacacctaaatggtatatataaaagaaactgttaaacagctttgtatgagttgttaatttgaattggccctcgataatcatgaaaattaagaacataattgtacctagtactaataaatgtgtatgtgtaacatttaattgagaaaaaccgctaaaatcttccgcttttgtcatttctctataaaatagaccacttaataaccctaatagtgtatagagcgctgaactatacattaatcttttcattttaattccccctatttttaattacgagataagtatagcggtagtttatgaactgagtatgaacttacaacaaaaaaattaatgaagtactttacaataaactcaatttattagatggtggagggacgaaaaaggattttagaaaaataaattaatatatttttattttgataagtaataattaataatatcttggaaatcattgttaagtattgttgtaatacaatcgtcattcataaaatcttcatagtttttatcaagaatttcttcatcttcgatagatgtgaaatgattagctaacccttttataatttaagtgtaatttgtgaatctaaac

SEQ ID NO: 20 - DNA, 1081 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatc

SEQ ID NO: 21 - DNA, 1054 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatcaacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgtggagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaa

SEQ ID NO: 22 - DNA, 4353 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaattaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactacgcactatcattcagcaaaatgacattcccacatcaaatgatgcgggttgtgttaattgagcaagtgtatagagcatttaagattatgcgtggagaagcatatcataaatgatgcggttttttcagccgcttcataaagggattttgaatgtatcagaacatatgaggtttatgtgaattgctgttatgtttttaagaagcatatcataaatgatgcggttttttcagccgcttcataaagggattttgaatgtatcagaacatatgaggtttatgtgaattgctgttatgtttttaagaagcatatcataagtgatgcggtttttattaattagttgctaaaaaatgaagtatgcaatattaattattattaaattttgatatatttaaagaaagattaagtttagggtgaatgaatggcttatcaaagtgaatatgcattagaaaatgaagtacttcaacaacttgaggaattgaactatgaaagagtaaatatacataatattaaattagaaattaatgaatatctcaaagaactaggagtgttgaaaaatgaataagcagacaaatactccagaactaagatttccagagtttgatgaggaatggaaaaaaaggaaattaggtgaagtagtaaattataaaaatggtggttcatttgaaagtttagtgaaaaaccatggtgtatataaactcataactcttaaatctgttaatacagaaggaaagttgtgtaattctggaaaatatatcgatgataaatgtgttgaaacattgtgtaatgatactttagtaatgatactgagcgagcaagcaccaggactagttggaatgactgcaattatacctaataataatgagtatgtactaaatcaacgagtagcagcactagtgcctaaacaatttatagatagtcaatttctatctaagttaattaatagaaaccagaaatatttcagtgtgagatctgctggaacaaaagtgaaaaatatttctaaaggacatgtagaaaactttaattttttatctcctaattacactgaacaacaaaaaataggtaatttcttcagcaaactcgaccgccagattgagttagaagaagagaaacttgaactcttagagcaacaaaagcgtggatatattcagaagattttttctcaagatttaagatttaaagatgaaaatggaaacagttatcctgattggtctattaaaaagattgaagatatttctaaagttaataaagggtttactccaaatacaaaaaatgataaatactgggatgaattaaatgaaaattggttatctatagcaggtatgacacagaaatatttgtataaaggaaataaaggaattactgaaaaaggtgcatcaaagcatgtaaaagtagataaagatactctaataatgagttttaaattgactttaggtaagttagctatagtaaaagagcctatctatacaaatgaagctatatgccatttcgtatggaaagaaagtaatgttaatactgagtatatgtactactatttaaattctataaatataagtacttttggtgcacaggcagttaaaggagtaacattaaataacgatgcaattaatagtattatagtaaagttaccagtgatacaagaacaaaataaaatagcatactttttcaataaattagataaattaattgaaaaacaatcttctaaagtagaattattaaaacaacgcaaacaaggatttttacagaaaatgtttgtttaattcttataaagttctattatgtaaaatattaaatagagataacattatgaaagcgagcccaagacataaagtttttgaataaataaaaaagataatttctatcaaattaatatagaaattgtcttttttataaattttttgattatttttagctgattgagctgttacttttcttataataagtgctattagcacaaatcctagttctcttttggctttgtttattcctcttacggacattcgagtgaaacccattttaattttattagaagtaatttaggtttgaacccacctaaataaatatatgagttatttttttatgctacaaaatatattcagatttcaataatgacataaaataggcatctttatatttacctttagtgtagaattgctctttgagtaatccttctgttttaaatccttgtgactcgtatatatgcacagcttttttgttatctgtatcaacatatagataaattttgtgcatgtttaatatatcgaatgcataatttatcgctttttcgaatgcgaattttgcataacctttaccactgaactcaggtttaataattatttgtatttcacaattacgatggatgtaattaatttctactaattcaacaatacctacgacttgattttcatcttcaacaataaaacgtctctctgattcatctaataaatgcttatcaaataaatattgaagttccgttaaggattcatatggttcttcaaaccaataagacataatagaatattcattatttaattcatgaacaaaaagtaaatcactatactctaatgctcttagtttcataattccactcccaaaattttctcatatatttgcattataaatataaataacgaataagtcatcattcactgtgaatactctattttaacaattcaccacatactaattctcattttcttgttattctcgatttattactcttactatgaaacctataaaattctcacatttgtttgtattaagaataaatacgtcgatagtaacaataaaaaaataaataataaagcatccctcaccgtaaaagtgaaggatgctctagttttattgaaatatacatttcattttgttaaataattattaataatattttgaaaatcattattacgtgaaatcttcatagattttatcaagtatttctttgccttcaattgctgtgaagtgatgtaccaatctatttttacaatcatatgtaattttgtgacgctaggtaattagtaattgttcgtcagtctgattgtatagtatcaagtttcatagataatactctttgattttaatgtccactttgacgtgctttaagattgagtatatacataatgtcattgtggaatgttaaaaatcctacaaatgtttattcatctgcaggatttttaaatctccaagaataaaaatcatcataggacaactggattattgtttggataaataacgtaaacaataattaggtactattatttatttttgtttattctttttcctaacaaaataaagaaaagaataaacgcaattgttaaaaatatgtgtcctaaaccagcaataccagcaatagcaggacttacacttagatctttaatggtagaaataccgttcacgaattgcattgccacagtaacaagcacacctaaatggtatatataaaagaaactgttaaacagctttgtatgagttgttaatttgaattggccctcgataatcatgaaaattaagaacataattgtacctagtactaataaatgtgtatgtgtaacatttaattgagaaaaaccgctaaaatcttccgcttttgtcatttctctataaaatagaccacttaataaccctaatagtgtatagagcgctgaactatacattaatcttttcattttaattccccctatttttaattacgagataagtatagcggtagtttatgaactgagtatgaacttacaacaaaaaaattaatgaagtactttacaataaactcaatttattagatggtggagggacgaaaaaggattttagaaaaataaattaatatatttttattttgataagtaataattaataatatcttggaaatcattgttaagtattgttgtaatacaatcgtcattcataaaatcttcatagattttatcaagaatttcttcatcttcgatagatgtgaaatgattagctaacccttttataatttaagtgtaatttgtgaatctaaac

SEQ ID NO: 23 - DNA, 2031 bp, Staphylococcus aureus
accattttagctgtagggaaactaaaagagaaatattggaagcaagccatagcagaatatgaaaaacgtttaggcccatacaccaagatagacatcatagaagttccagacgaaaaagcaccagaaaatatgagtgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgttttcgtcattggcggatcaaacggcctgcacaaggacgtcttacaacgcagtaactacgcactatcattcagcaaaatgacattcccacatcaaatgatgcgggttgtgttaattgaacaagtgtacagagcatttaagattatgcgaggagaagcgtatcacaaataaaactaaaaaatagattgtgtataatataaaaggagcggatttatattaaaactttgaattcaaaaattattgaaagggaagctaccttagaaattgaatctatggcaactaatacattgaaaataaacccggatattaattcaaacgatacaaaaatgtctttcgatggagaattggaagtgtatgattctgaaaatttgagtaaaaaaaatttcgttggaaaaatacaagttcaagttaaaggaaaggaagtagctaaaagaggaggtaaggttattcatcgaagtaatgtcaaaatgaatgatttaaaggcataccaacgagaaggtggtgtgtattactttgtcgtgtatttaatcgttgagaataaaaaagttgttgagaagcaggtttatggtaaacaattacatcaattagatttacaatttttactgcaaaaaaagcagaaaagcgtcactataaaaatgtatgaaattgaaaatgaaaaaattttatataataattgcgtaaaatatataaatgaaaagagattacaaaatcaagtaggtcaagttaaagtaaagaacatagaaaaagctttatcatatatagctacacctgaaaatattgtgatggatcatagaggtttaccattaaatgatttttatggctatataaaaattaattcatctgaattagatgtaactataccagatggagtattaagtatggaaaaagtaaaaagagtgaacaaaaagcagataataaaagaaggcaagttattatttgaaggtatggtaggtattgaaacttcaaaggagtccatctctataactatagatgatattttcaaaattcaaacatttgaaagtgataacaaaagtatatatacaatgttaccatttaaaaaattaaatatagccgaacaatcttttaatgttataaacgaattgtcgaaaggcggcgaattctttttggatcaaatccaattagtaatccaaccttttgaaattaatattattgaaataaaagaaacaataaataagttgaatattaaattatcagagtatagtaacttgctttcgtttgatgtgagtctaaaatctacagaatttgataagcagatgaatgagataaaaggtttattagaattattggaatataaaaattttaaagattttaaaatgcataataatggatactataaaatgaagttttgtggaaaatttatattattatttaaagacaatacatcattgtataatgtctactctaatgactttgtagatagatttgaggctgttacaaaagaaagagttgtacagatgccaattgtttacacattaacaagagatatgattgtagatgtactgaattttgatataaatgttattaaagaatgtattgaatcagataaaattgctattcaatctgacattaaatgggagaaattaaataactttgcattagaattaatcgcagcctatgatgaaactcaaagaaccgatttattagaattagctgaatatgtattaaataatttgctgaattttgataatgataaaatatttatgaacttaa

SEQ ID NO: 24 - DNA, 818 bp, Staphylococcus aureus
aatattggaagcaagccatagcagaatatgaaaaacgtttaggcccatacaccaagatagacatcatagaagttccagacgaaaaagcaccagaaaatatgagcgacaaagaaattgagcaagtgtatagagcatttaagattatgcgtggagaagcatatcataaatgatgcggttttttcagccgcttcataaagggattttgaatgtatcagaacatatgaggtttatgtgaattgctgttatgtttttaagaagcttatcataagtaatgaggttcatgatttttgacatagttagcctccgcagtctttcatttcaagtaaataatagcgaaatattctttatactgaatacttatagtgaagcaaagttctagctttgagaaaattctttctgcaactaaatatagtaaattacggtaaaatataaataagtacatattgaagaaaatgagacataatatattttataataggagggaatttcaaatgatagacaactttatgcaggtccttaaattaattaaagagaaacgtaccaataatgtagttaaaaaatctgattgggataaaggtgatctatataaaactttagtccatgataagttacccaagcagttaaaagtgcatataaaagaagataaatattcagttgtagggaaggttgctactgggaactatagtaaagttccttggatttcaatatatgatgagaatataacaaaagaaacaaaggatggatattatttggtatatctttttcatccggaaggagaaggcatatacttatcttgaatcaaggatggtcaaagataa

SEQ ID NO: 25 - DNA, 1073 bp, Staphylococcus aureus
tattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaggcgtatcataagtgatgcttgttagaatgatttttaacaatatgaaatagctgtggaagcttaaacaatttgtttatctaagtacttatttaataattgattgaactgtgattggcaccaggctgtctggtaaattgagaagttgggttttggagcgtataaatgatagaattaatataaaattcaatttgaggagtaggagattatgtcgaatataaaaacaacactagagacgtccgtaggactagaaaaagacaacgataagctatttgattatataactgaattagagattcaaaacacgcctgaaaaccgggaagcaaaagttgttattgaagaaaggttacataaagaatataaatatgaattagatcaaatgacaccagagtatggaatacaaaaaggcagtgttagaataggtcatgcagatgttgtaatatttcatgattctaaagataaatctcaagagaatattaaaataatagtagagtgtaaaagaaagaatcgcagggatggtattgaacaattaaaaacatatcttgcagggtgtgagtctgcagaatacggcgtttggtttaatggagaagatatagtatatataaaacgattgaaaaaagcaccacattggaaaacagtatttaatataccga

SEQ ID NO: 26 - DNA, 756 bp, Staphylococcus aureus
tgacattcccacatcaaatgatgcgggttgtgttaattgagcaagtgtatagagcatttaagattatgcgtggagaagcgtatcacaaataaaactaaaaaataggttgcgcataatataattagaaaggaattagacataaattaggagtccttcacagaatagcgaaggactcccattaaatatattatggtgtaaagaaatcacaaatcaatatatatacttaataccatatattaacttgtactattataaagtacgacatcagtattaggtatcactttgaacacatgaatttcattatcacttttattattcacaaaaaattttccaattctcaattactgaattatgtgtatacatgttgttaaaaattaataaaggatatttatgtttgtttaaagcatatcacaagtgatgcggttttttataaagatttacttgttagtgattttgataaaaatgcttaatactatttcaataatatgtatttaaaaattagattaatagtatttaacttcaaatggcctcgtataaactcatagcaaattaacgtaaatcaatgaaataaaatgaaaacaatttcaagaatacattataaacataaagtatacaaaaaataaatgagcgtatttgtttaaacgtatacactcatttttattaaattaatttattatattttacgattgttatttatgaaattaacaaattccatttttgatagtgaaattaaaagctttatcacttattattgat

SEQ ID NO: 27 - DNA, 771 bp, Staphylococcus aureus
tgacattcccacatcaaatgatgcgggttgtgttaattgagcaagtgtatagagcatttaagattatgcgtggagaagcgtatcacaaataaaactaaaaaataggttgcgcataatataattagaaaggaattagacataaattaggagtccttcacagaatagcgaaggactcccattaaatatattatggtgtaaagaaatcacaaatcaatatatatacttaataccatatattaacttgtactattataaagtacgacatcagtattaggtatcactttgaacacatgaatttcattatcacttttattattcacaaaaaattttccaattctcaattactgaattatgtgtatacatgttgttaaaaattaataaaggatatttatgtttgtttaaagcatatcacaagtgatgcggttttttataaagatttacttgttagtgattttgataaaaatgcttaatactatttcaataatatgtatttaaaaattagattaatagtatttaacttcaaatggcctcgtataaactcatagcaaattaacgtaaatcaatgaaataaaatgaaaacaatttcaagaatacattataaacataaagtatacaaaaaataaatgagcgtatttgtttaaacgtatacactcatttttattaaattaatttattatattttacgattgttatttatgaaattaacaaattccatttttgatagtgaaattaaaagctttatcacttattattgataattttgactgcatc

SEQ ID NO: 28 - DNA, 680 bp, Staphylococcus aureus
ttcagcaaaatgacattcccacatcaaatgatgcgggttgtgttaattgaacaagtgtacagagcatttaagattatgcgtggagaagcgtatcataagtagcggaggagttttttaccttgtgacttatcataaagtacgatgtttatgtaagtgattatcattatttaagcaggtttttcaaattaaataataacaagaataaaatgcacttagcgacattgaaatttattaatctagtaaactaatagatttatagaaaattttatttgcaaggggataattttgaaaagtagtattttctatctttccataatacattgtaattacaacggaggggatattgtgatgaagtgtatagataaaacgtgggttagctattataaagaattagctgataagttaacagattatcaaaataaacgttatgaattaattgaaatagtgaaggaagtatataaaaaaacgggaataaaattccctactttagcaagtgataatgtattgatggacatagatccttttacaatatttgcattatttaataaaaattccatgagagaaactaataaggtaaaaatattaacagaattagcttcggaattgaatattaagtccaaaattccgtcagtttttgacagtattccaacagtcaataatctgaatgctacatattataa

SEQ ID NO: 29 - DNA, 1119 bp, Staphylococcus aureus
gacattcccacatcaaatgatgcgggttgtgttaattgagcaagtgtatagagcatttaagattatgcgtggagaggcgtatcataagtaaaactaaaaaattctgtatgaggagataataatttggagggtgttaaatggtggacattaaatccacgttcattcaatatataagatatatcacgataattgcgcatataacttaagtagtagctaacagttgaaattaggccctatcaaattggtttatatctaaaatgattaatatagaatgcttctttttgtccttattaaattataaaagtaactttgcaatagaaacagttatttcataatcaacagtcattgacgtagctaagtaatgataaataatcataaataaaattacagatattgacaaaaaatagtaaatataccaatgaagtttcaaaagaacaattccaagaaattgagaatgtaaataataaggtcaaagaattttattaagatttgaaagagtatcaatcaagaaagatgtagttttttaataaactatttggaaaataattatcataatttaaaaactgacaatttgcgagactcataaaatgtaataatggaaatagatgtaaaatataattaaggggtgtaatatgaagattaatatttataaatctatttataattttcaggaaacaaatacaaattttttagagaatctagaatctttaaatgatgacaattatgaactgcttaatgataaagaacttgttagtgattcaaatgaattaaaattaattagtaaagtttatatacgtaaaaaagacaaaaaactattagattggcaattattaataaagaatgtatacctagatactgaagaagatgacaatttattttcagaatccggtcatcattttgatgcaatattatttctcaaagaagatactacattacaaaataatgtatatattataccttttggacaagcatatcatgatataaataatttgattgattatgacttcggaattgattttgcagaaagagcaatcaaaaatgaagacatagttaataaaaatgttaatttttttcaacaaaacaggcttaaagagattgttaattatagaaggaatagtg

SEQ ID NO: 30 - DNA, 24 bp, Artificial Sequence
  Synthesized Oligonucleotide
caaattgtagcattcttgaactat

SEQ ID NO: 31 - DNA, 26 bp, Artificial Sequence
  Synthesized Oligonucleotide
ctattgttccacaaattatgattact

SEQ ID NO: 32 - DNA, 24 bp, Artificial Sequence
  Synthesized Oligonucleotide
ctcccatttcttccaaaaaatata

SEQ ID NO: 33 - DNA, 26 bp, Artificial Sequence
  Synthesized Oligonucleotide
cttcttttcttgttattctttcttct

SEQ ID NO: 34 - DNA, 24 bp, Artificial Sequence
  Synthesized Oligonucleotide
taatttcctttttttccattcctc

SEQ ID NO: 35 - DNA, 24 bp, Artificial Sequence
  Synthesized Oligonucleotide
gagcggatttatattaaaactttg

SEQ ID NO: 36 - DNA, 24 bp, Artificial Sequence
  Synthesized Oligonucleotide
gttgccatagattcaatttctaag

SEQ ID NO: 37 - DNA, 20 bp, Artificial Sequence
  Synthesized Oligonucleotide
cgaggagaagcgtatcacaa

SEQ ID NO: 38 - DNA, 27 bp, Artificial Sequence
  Synthesized Oligonucleotide
agttgccatagattcaatttctaaggt

SEQ ID NO: 39 - DNA, 29 bp, Artificial Sequence
  Synthesized Oligonucleotide
gtcaaaaatcatgaacctcattacttatg

SEQ ID NO: 40 - DNA, 19 bp, Artificial Sequence
  Synthesized Oligonucleotide
caccctgcaagatatgttt

SEQ ID NO: 41 - DNA, 22 bp, Artificial Sequence
  Synthesized Oligonucleotide
cgtggagaggcgtatcataagt

SEQ ID NO: 42 - DNA, 22 bp, Artificial Sequence
  Synthesized Oligonucleotide
gctccaaaacccaacttctcaa

SEQ ID NO: 43 - DNA, 25 bp, Artificial Sequence
  Synthesized Oligonucleotide
aatggaatttgttaatttcataaat

SEQ ID NO: 44 - DNA, 23 bp, Artificial Sequence
  Synthesized Oligonucleotide
ttccgaagctaattctgttaata

SEQ ID NO: 45 - DNA, 25 bp, Artificial Sequence
  Synthesized Oligonucleotide
ttccgaagtcataatcaatcaaatt

SEQ ID NO: 46 - DNA, 23 bp, Artificial Sequence
  Synthesized Oligonucleotide
gccaaaatcaaaccacaatccac

SEQ ID NO: 47 - DNA, 23 bp, Artificial Sequence
  Synthesized Oligonucleotide
gccaaaatcaaaccacaatcaac

SEQ ID NO: 48 - DNA, 23 bp, Artificial Sequence
  Synthesized Oligonucleotide
accacgaatgtttgctgctaatg

SEQ ID NO: 49 - DNA, 36 bp, Artificial Sequence
  Synthesized Oligonucleotide
cgcttgccacatcaaatgatgcgggttgtgcaagcg

SEQ ID NO: 50 - DNA, 36 bp, Artificial Sequence
  Synthesized Oligonucleotide
cccaccccacatcaaatgatgcgggttgtgggtggg

SEQ ID NO: 51 - DNA, 37 bp, Artificial Sequence
  Synthesized Oligonucleotide
cccgcgcgtagttactgcgttgtaagacgtccgcggg

SEQ ID NO: 52 - DNA, 48 bp, Artificial Sequence
  Synthesized Oligonucleotide
cgaccggattcccacatcaaatgatgcgggttgtgttaattccggtcg

SEQ ID NO: 53 - DNA, 37 bp, Artificial Sequence
  Synthesized Oligonucleotide
cccgcgcrtagttactrcgttgtaagacgtccgcggg

SEQ ID NO: 54 - DNA, 29 bp, Artificial Sequence
  Synthesized Oligonucleotide
ccccgtagttactgcgttgtaagacgggg

SEQ ID NO: 55 - DNA, 37 bp, Artificial Sequence
  Synthesized Oligonucleotide
cccgcgcatagttactgcgttgtaagacgtccgcggg

SEQ ID NO: 56 - DNA, 37 bp, Artificial Sequence
  Synthesized Oligonucleotide
cccgcgcgtagttactacgttgtaagacgtccgcggg

SEQ ID NO: 57 - DNA, 19 bp, Artificial Sequence
  Synthesized Oligonucleotide
ggatcaaacggcctgcaca

SEQ ID NO: 58 - DNA, 21 bp, Artificial Sequence
  Synthesized Oligonucleotide
atcaaatgatgcgggttgtgt

SEQ ID NO: 59 - DNA, 19 bp, Artificial Sequence
  Synthesized Oligonucleotide
tcattggcggatcaaacgg

SEQ ID NO: 60 - DNA, 22 bp, Artificial Sequence
  Synthesized Oligonucleotide
acaacgcagtaactacgcacta

SEQ ID NO: 61 - DNA, 22 bp, Artificial Sequence
  Synthesized Oligonucleotide
taactacgcactatcattcagc

SEQ ID NO: 62 - DNA, 22 bp, Artificial Sequence
  Synthesized Oligonucleotide
acatcaaatgatgcgggttgtg

SEQ ID NO: 63 - DNA, 22 bp, Artificial Sequence
  Synthesized Oligonucleotide
tcaaatgatgcgggttgtgtta

SEQ ID NO: 64 - DNA, 24 bp, Artificial Sequence
  Synthesized Oligonucleotide
caaatgatgcgggttgtgttaatt

SEQ ID NO: 65 - DNA, 29 bp, Artificial Sequence
  Synthesized Oligonucleotide
gtcaaaaatcatgaacctcattacttatg

SEQ ID NO: 66 - DNA, 28 bp, Artificial Sequence
  Synthesized Oligonucleotide
ctatgtcaaaaatcatgaacctcattac

SEQ ID NO: 67 - DNA, 23 bp, Artificial Sequence
  Synthesized Oligonucleotide
ggaggctaactatgtcaaaaatc

SEQ ID NO: 68 - DNA, 27 bp, Artificial Sequence
  Synthesized Oligonucleotide
gctgaaaaaaccgcatcatttrtgrta

SEQ ID NO: 69 - DNA, 27 bp, Artificial Sequence
  Synthesized Oligonucleotide
gctgaaaaaaccgcatcatttatgata

SEQ ID NO: 70 - DNA, 29 bp, Artificial Sequence
  Synthesized Oligonucleotide
atttcatatatgtaattcctccacatctc

SEQ ID NO: 71 - DNA, 29 bp, Artificial Sequence
  Synthesized Oligonucleotide
tttagttttatttatgatacgcttctcca

SEQ ID NO: 72 - DNA, 25 bp, Artificial Sequence
  Synthesized Oligonucleotide
ctctataaacatcgtatgatattgc

SEQ ID NO: 73 - DNA, 29 bp, Artificial Sequence
  Synthesized Oligonucleotide
caaatattatctcgtaatttaccttgttc

SEQ ID NO: 74 - DNA, 29 bp, Artificial Sequence
  Synthesized Oligonucleotide
ctctgctttatattataaaattacggctg

SEQ ID NO: 75 - DNA, 22 bp, Artificial Sequence
  Synthesized Oligonucleotide
ttcgttccctccattaactgtc

SEQ ID NO: 76 - DNA, 22 bp, Artificial Sequence
  Synthesized Oligonucleotide
tcaccgtctttcttttgacctt

SEQ ID NO: 77 - DNA, 27 bp, Artificial Sequence
  Synthesized Oligonucleotide
cactttttattcttcaaagatttgagc

SEQ ID NO: 78 - DNA, 28 bp, Artificial Sequence
  Synthesized Oligonucleotide
atggaaattcttaatctttacttgtacc

SEQ ID NO: 79 - DNA, 23 bp, Artificial Sequence
  Synthesized Oligonucleotide
cagcaattcwcataaacctcata

SEQ ID NO: 80 - DNA, 27 bp, Artificial Sequence
  Synthesized Oligonucleotide
acaaactttgaggggatttttagtaaa

SEQ ID NO: 81 - DNA, 24 bp, Artificial Sequence
  Synthesized Oligonucleotide
tgataagccattcattcaccctaa

SEQ ID NO: 82 - DNA, 24 bp, Artificial Sequence
  Synthesized Oligonucleotide
tagttactgtgttgtaagacgtcc

SEQ ID NO: 83 - DNA, 26 bp, Artificial Sequence
  Synthesized Oligonucleotide
ctactatgaactgtgcaatttgttct

SEQ ID NO: 84 - DNA, 26 bp, Artificial Sequence
  Synthesized Oligonucleotide
gcaattcacataaacctcatatgttc

SEQ ID NO: 85 - DNA, 24 bp, Artificial Sequence
  Synthesized Oligonucleotide
acctcatatgttctgatacattca

SEQ ID NO: 86 - DNA, 22 bp, Artificial Sequence
  Synthesized Oligonucleotide
gcaattcacataaacctcatat

SEQ ID NO: 87 - DNA, 25 bp, Artificial Sequence
  Synthesized Oligonucleotide
cataacagcaattcacataaacctc

SEQ ID NO: 88 - DNA, 22 bp, Artificial Sequence
  Synthesized Oligonucleotide
taacagcaattcacataaacct

SEQ ID NO: 89 - DNA, 27 bp, Artificial Sequence
  Synthesized Oligonucleotide
cgctattatttacttgaaatgaaagac

SEQ ID NO: 90 - DNA, 21 bp, Artificial Sequence
  Synthesized Oligonucleotide
cttgaaatgaaagactgcgga

SEQ ID NO: 91 - DNA, 54 bp, Artificial Sequence
  Synthesized Oligonucleotide
ttgcttcactataagtattcagtataaagaatttacttgaaatgaaagactgcg

SEQ ID NO: 92 - DNA, 24 bp, Artificial Sequence
  Synthesized Oligonucleotide
atttacttgaaatgaaagactgcg

SEQ ID NO: 93 - DNA, 29 bp, Artificial Sequence
  Synthesized Oligonucleotide
aaagaatatttcgctattatttacttgaa

SEQ ID NO: 94 - DNA, 29 bp, Artificial Sequence
  Synthesized Oligonucleotide
tcagtataaagaatatttcgctattattt

SEQ ID NO: 95 - DNA, 20 bp, Artificial Sequence
  Synthesized Oligonucleotide
tgaaatgaaagactgcggag

SEQ ID NO: 96 - DNA, 27 bp, Artificial Sequence
  Synthesized Oligonucleotide
aacctcatatgttctgatacattcaaa

SEQ ID NO: 97 - DNA, 28 bp, Artificial Sequence
  Synthesized Oligonucleotide
tatgtcaaaaatcatgaacctcattact

SEQ ID NO: 98 - DNA, 25 bp, Artificial Sequence
  Synthesized Oligonucleotide
cataacagcaattcacataaacctc

SEQ ID NO: 99 - DNA, 17 bp, Artificial Sequence
  Synthesized Oligonucleotide
gactgcggaggctaact

SEQ ID NO: 100 - DNA, 18 bp, Artificial Sequence
  Synthesized Oligonucleotide
atccctttatgaagcggc

SEQ ID NO: 101 - DNA, 20 bp, Artificial Sequence
  Synthesized Oligonucleotide
tgaaatgaaagactgcggag

SEQ ID NO: 102 - DNA, 30 bp, Artificial Sequence
  Synthesized Oligonucleotide
gcaaggtataatccaatatttcatatatgt

SEQ ID NO: 103 - DNA, 29 bp, Artificial Sequence
  Synthesized Oligonucleotide
agttccataatcaatataatttgtacagt

SEQ ID NO: 104 - DNA, 23 bp, Artificial Sequence
  Synthesized Oligonucleotide
acatcgtatgatattgcaaggta

SEQ ID NO: 105 - DNA, 26 bp, Artificial Sequence
  Synthesized Oligonucleotide
ctttcattctttcttgattccattag

SEQ ID NO: 106 - DNA, 27 bp, Artificial Sequence
  Synthesized Oligonucleotide
cactctataaacatcgtatgatattgc

SEQ ID NO: 107 - DNA, 30 bp, Artificial Sequence
  Synthesized Oligonucleotide
ttcttaatttaattgtagttccataatcaa

SEQ ID NO: 108 - DNA, 30 bp, Artificial Sequence
  Synthesized Oligonucleotide
aattatacacaacctaatttttagttttat

SEQ ID NO: 109 - DNA, 29 bp, Artificial Sequence
  Synthesized Oligonucleotide
aatttttagttttatttatgatacgcttc

SEQ ID NO: 110 - DNA, 30 bp, Artificial Sequence
  Synthesized Oligonucleotide
acacaacctaatttttagttttatttatga

SEQ ID NO: 111 - DNA, 30 bp, Artificial Sequence
  Synthesized Oligonucleotide
tttattaaacactctataaacatcgtatga

SEQ ID NO: 112 - DNA, 31 bp, Artificial Sequence
  Synthesized Oligonucleotide
tcacatctcattaaatttttaaattatacac

SEQ ID NO: 113 - DNA, 31 bp, Artificial Sequence
  Synthesized Oligonucleotide
ccacatctcattaaatttttaaattatacac

SEQ ID NO: 114 - DNA, 30 bp, Artificial Sequence
  Synthesized Oligonucleotide
atattatacacaatccgttttttagtttta

SEQ ID NO: 115 - DNA, 29 bp, Artificial Sequence
  Synthesized Oligonucleotide
acacaatccgttttttagttttatttatg

SEQ ID NO: 116 - DNA, 30 bp, Artificial Sequence
  Synthesized Oligonucleotide
ttctaatttatttaacataaaatcaatcct

SEQ ID NO: 117 - DNA, 33 bp, Artificial Sequence
  Synthesized Oligonucleotide
caatcctttttatatttaaaatatattatacac

SEQ ID NO: 118 - DNA, 22 bp, Artificial Sequence
  Synthesized Oligonucleotide
aagtcgctttgcctttgggtca

SEQ ID NO: 119 - DNA, 26 bp, Artificial Sequence
  Synthesized Oligonucleotide
tacaaagtcgctttgcctttgggtca

SEQ ID NO: 120 - DNA, 19 bp, Artificial Sequence
  Synthesized Oligonucleotide
ggccgtttgatccgccaat

SEQ ID NO: 121 - DNA, 21 bp, Artificial Sequence
  Synthesized Oligonucleotide
aagtcgctttgcccttgggta

SEQ ID NO: 122 - DNA, 20 bp, Artificial Sequence
  Synthesized Oligonucleotide
aagtcgctttgcccttgggt

SEQ ID NO: 123 - DNA, 22 bp, Artificial Sequence
  Synthesized Oligonucleotide
aagtcgctttgcccttgggtca

SEQ ID NO: 124 - DNA, 19 bp, Artificial Sequence
  Synthesized Oligonucleotide
aagtcgctttgcccttggg

SEQ ID NO: 125 - DNA, 20 bp, Artificial Sequence
  Synthesized Oligonucleotide
caagaattgaaccaacgcat

SEQ ID NO: 126 - DNA, 30 bp, Artificial Sequence
  Synthesized Oligonucleotide
caatgacgaatacatagtcgctttgccctt

SEQ ID NO: 127 - DNA, 21 bp, Artificial Sequence
  Synthesized Oligonucleotide
cgtttgatccgccaatgacga

SEQ ID NO: 128 - DNA, 22 bp, Artificial Sequence
  Synthesized Oligonucleotide
gccaatccttcggaagatagca

SEQ ID NO: 129 - DNA, 18 bp, Artificial Sequence
  Synthesized Oligonucleotide
attaacacaacccgcatc

SEQ ID NO: 130 - DNA, 19 bp, Artificial Sequence
  Synthesized Oligonucleotide
gtcgctttgcccttgggtc

SEQ ID NO: 131 - DNA, 20 bp, Artificial Sequence
  Synthesized Oligonucleotide
tcgctttgcccttgggtcat

SEQ ID NO: 132 - DNA, 19 bp, Artificial Sequence
  Synthesized Oligonucleotide
ggccgtttgatccgccaat

SEQ ID NO: 133 - DNA, 22 bp, Artificial Sequence
  Synthesized Oligonucleotide
gtccttgtgcaggccgtttgat

SEQ ID NO: 134 - DNA, 24 bp, Artificial Sequence
  Synthesized Oligonucleotide
cttgggtcatgcgttggttcaatt

SEQ ID NO: 135 - DNA, 27 bp, Artificial Sequence
  Synthesized Oligonucleotide
cgaatacaaagtcgctttgcccttggg

SEQ ID NO: 136 - DNA, 20 bp, Artificial Sequence
  Synthesized Oligonucleotide
atgcgttggttcaattcttg

SEQ ID NO: 137 - DNA, 20 bp, Artificial Sequence
  Synthesized Oligonucleotide
gcgttggttcaattcttggg

SEQ ID NO: 138 - DNA, 20 bp, Artificial Sequence
  Synthesized Oligonucleotide
acccaagggcaaagcgactt

SEQ ID NO: 139 - DNA, 24 bp, Artificial Sequence
  Synthesized Oligonucleotide
ggtaatgcgttggttcaattcttg

SEQ ID NO: 140 - DNA, 25 bp, Artificial Sequence
  Synthesized Oligonucleotide
acaaagtcgctatgcccttgggtca

SEQ ID NO: 141 - DNA, 29 bp, Artificial Sequence
  Synthesized Oligonucleotide
ctttccttgtatttctaatgtaatgactg

SEQ ID NO: 142 - DNA, 27 bp, Artificial Sequence
  Synthesized Oligonucleotide
ttgatgtgggaatgtcattttgctgaa

SEQ ID NO: 143 - DNA, 25 bp, Artificial Sequence
  Synthesized Oligonucleotide
gcgttggttcaattcttgggccaat

SEQ ID NO: 144 - DNA, 29 bp, Artificial Sequence
  Synthesized Oligonucleotide
gttggttcaattcttgggccaatccttcg

SEQ ID NO: 145 - DNA, 26 bp, Artificial Sequence
  Synthesized Oligonucleotide
cgaatacaaagtcgctttgcccttgg

SEQ ID NO: 146 - DNA, 29 bp, Artificial Sequence
  Synthesized Oligonucleotide
gccaatgacgaatacaaagtcgctttgcc

SEQ ID NO: 147 - DNA, 25 bp, Artificial Sequence
  Synthesized Oligonucleotide
tgggccaatccttcggaagatagca

SEQ ID NO: 148 - DNA, 20 bp, Artificial Sequence
  Synthesized Oligonucleotide
atgcgttggttcgattcttg

SEQ ID NO: 149 - DNA, 21 bp, Artificial Sequence
  Synthesized Oligonucleotide
catgcgttggttcgattcttg

SEQ ID NO: 150 - DNA, 19 bp, Artificial Sequence
  Synthesized Oligonucleotide
aagtcgctttgcccttggg

SEQ ID NO: 151 - DNA, 21 bp, Artificial Sequence
  Synthesized Oligonucleotide
catgcgttggttcgattcttg

SEQ ID NO: 152 - DNA, 23 bp, Artificial Sequence
  Synthesized Oligonucleotide
aagtcgctttgcccttgggtcat

SEQ ID NO: 153 - DNA, 25 bp, Artificial Sequence
  Synthesized Oligonucleotide
tgctcaattaacacaacccgcatca

SEQ ID NO: 154 - DNA, 33 bp, Artificial Sequence
  Synthesized Oligonucleotide
gccgcgctgctcaattaacacaacccgcgcggc

SEQ ID NO: 155 - DNA, 32 bp, Artificial Sequence
  Synthesized Oligonucleotide
gccgcgcatgcgttggttcaattctgcgcggc

SEQ ID NO: 156 - DNA, 2123 bp, Staphylococcus aureus
ctccatatcacaaaaattataacattattttgacataaatactacatttgtaatatactacaaatgtagtcttatataaggaggatattgatgaaaaagataaaaattgttccacttattttaatagttgtagttgtcgggtttggtatatatttttatgcttcaaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactatatcaaacaacaaatggatcaaaattgggtacaagatgataccttcgttccacttaaaaccgttaaaaaaatggatgaatatttaagtgatttcgcaaaaaaatttcatcttacaactaatgaaacagaaagtcgtaactatcctctagaaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaactgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataacaaaacagtgaagcaatccgtaacga

SEQ ID NO: 157 - DNA, 1998 bp, Staphylococcus aureus
ttactgatctatatcaaattgagtttttccattatcatacaaatcatcataaacttttccagatatagtagcattatagctggccatccctttattttgaacgtctttaacattaatcgccattaacatattaggattatttttattatatgaaacaaaccaacctatttgtcttccagtttccccttgattcatttttaattctgctgtgccagatttaccaataattcgggcataatttttgtatatatcatccctatgtgttttattaactacacgttccataccattagttaatatatctatgtcttttttaggtataatatcttttttccatatttgagattttgttttacgtaaaacatgaggattttgtatatttccgttattttctaaagcactgtatattgataaaatttgtatagggtttactagtatctcgccttggccatatcctgaatctgctaataatatttcattttttaaattactatttgagatttgtgctttataaaagggataatcactcgggatattttcaccgattcccaaatcttgcataccttgctcaaattttttggctcctaatgctaatgcaatgcgggcaaaaaatatgttgtctgatgattctattgcttgctttaaatcgatattgccgtctactactttaaatcttgtgatattataattaccccatgatgcatctttttgccaacccttaccataaatatcaaaattagtatttttgtctagtttattttcttttaaggctataatagatgttaatattttttgggttgaacctggtgatgtagtgatttgaaatttgttgagcaaaggctcttttttattgttagttaatttacggtagtcattattgcttaatccattcatgaatggataaacatcgtacgatggggtacttaccaaagctaaaatttctccagtttttggttgtaatgctgtaccagatccatcgtcatttttcatatgtttataaatactttcttgtactctagcatctatagttaaatgaagatcttttccgttttcagcctttttctccaataatgtgtctaaaggtttattgtcataagtatttgcaatggataccttaaaaccatcagtgttttgcaattgtttatcatagaggcgttctaagcctttttttccaataacagtatttttgctatagtttctaaattgcttactttttaactcgtcagaattaattggacccacataacctaaaaggtgtactgttgcttcgttcaatggataaacacggctttttatagtgtttatttgtaaattgtatgatttaattaatttgtctatatattcatcttgtttatttatctttttaattggtacaaatgaatctggctgaacccatttttgattaactttattggttatagcttttgtatcaatttgtaagtcacgagcaatatcatcatatttttctttgggtgttttgttagggacaataccgatttcatatgtatttccagttttagctaattctataccatttctatcttttattttgcctcgctctgattttaatgtttctatattaattttctgtccatttttcaaaccaggtactattacgtctggtctccaatctaatttccaatgcttatcttcataaataaagtttaattgtgtattacgtcgtatagttccatattttgtatatatgttatatttaacatcaacttgctttttatcttttccagtttttttaatttcatgattagtaatttttaagttattgacacttaaatctttgtaaatttttttattcctatctacaatttcttcttctccatatgccagtttagatttttctgaactatttttatatacttcgttatagtttcctttttcaatagaactaattgttttctcaatatcgtcatctttgaataaccaagttattataatcataattagtaaaagaactagcacactaatataaatttttttcat

SEQ ID NO: 158 - DNA, 686 bp, Staphylococcus aureus
atgacagaatacttattaagtgctggcatatgtatggcaattgtttcaatattacttatagggatggctatcagtaatgtttcgaaagggcaatacgcaaagaggtttttctttttcgctactagttgcttagtgttaactttagttgtagtttcaagtctaagtagctcagcaaatgcatcacaaacagataacggcgtaaatagaagtggttctgaagatccaacagtatatagtgcaacttcaactaaaaaattacataaagaacctgcgacattaattaaagcgattgatggtgatacggttaaattaatgtacaaaggtcaaccaatgacattcagactattattagttgatacacctgaaacaaagcatcctaaaaaaggtgtagagaaatatggccctgaagcaagtgcatttacgaaaaaaatggtagaaaatgcaaataaaattgaagtcgagtttgacaaaggtcaaagaactgataaatatggacgtggcttagcgtatatttatgctgatggaaaaatggtaaacgaagctttagttcgtcaaggcttggctaaagttgcttatgtttataaacctaacaatacacatgaacaacttttaagaaaaagtgaagcacaagcgaaaaaagagaaattaaatatttggagcgaagacaacgctgattcaggtcaata

SEQ ID NO: 159 - DNA, 686 bp, Staphylococcus aureus; identical to SEQ ID NO: 158

SEQ ID NO: 160 - DNA, 442 bp, Staphylococcus aureus
gatcaatctttgtcggtacacgatattcttcacgactaaataaacgctcattcgcgattttataaatgaatgttgataacaatgttgtattatctactgaaatctcattacgttgcatcggaaacattgtgttctgtatgtaaaagccgtcttgataatctttagtagtaccgaagctggtcatacgagagttatattttccagccaaaacgatatttttataatcattacgtgaaaaaggtttcccttcattatcacacaaatattttagcttttcagtttctatatcaactgtagcttctttatccatacgttgaataattgtacgattctgacgcaccatcttttgcacacctttaatgttatttgttttaaaagcatgaataagtttttcaacacaacgatgtgaatcttctaagaagtcaccgtaaaatgaaggatc

SEQ ID NO: 161 - DNA, 24 bp, Artificial Sequence
  Synthesized Oligonucleotide
acagaatacttattaagtgctggc

SEQ ID NO: 162 - DNA, 27 bp, Artificial Sequence
  Synthesized Oligonucleotide
gtttcaatattacttatagggatggct

SEQ ID NO: 163 - DNA, 24 bp, Artificial Sequence
  Synthesized Oligonucleotide
ctgatggaaaaatggtaaacgaag

SEQ ID NO: 164 - DNA, 22 bp, Artificial Sequence
  Synthesized Oligonucleotide
atggaaaaatggtaaacgaagc

SEQ ID NO: 165 - DNA, 22 bp, Artificial Sequence
  Synthesized Oligonucleotide
tgctgagctacttagacttgaa

SEQ ID NO: 166 - DNA, 22 bp, Artificial Sequence
  Synthesized Oligonucleotide
gcatttgctgagctacttagac

SEQ ID NO: 167 - DNA, 25 bp, Artificial Sequence
  Synthesized Oligonucleotide
gttgttcatgtgtattgttaggttt

SEQ ID NO: 168 - DNA, 22 bp, Artificial Sequence
  Synthesized Oligonucleotide
ttattgacctgaatcagcgttg

SEQ ID NO: 169 - DNA, 30 bp, Artificial Sequence
  Synthesized Oligonucleotide
cgaaacattactgatagccatccctataag

SEQ ID NO: 170 - DNA, 40 bp, Artificial Sequence
  Synthesized Oligonucleotide
cgcaccgaaacattactgatagccatccctataaggtgcg

SEQ ID NO: 171 - DNA, 29 bp, Artificial Sequence
  Synthesized Oligonucleotide
acataagcaactttagccaagccttgacg

SEQ ID NO: 172 - DNA, 41 bp, Artificial Sequence
  Synthesized Oligonucleotide
cgcgcaacataagcaactttagccaagccttgacgtgcgcg

SEQ ID NO: 173 - DNA, 30 bp, Artificial Sequence
  Synthesized Oligonucleotide
aatctttgtcggtacacgatattcttcacg

SEQ ID NO: 174 - DNA, 30 bp, Artificial Sequence
  Synthesized Oligonucleotide
cgtaatgagatttcagtagataatacaaca

SEQ ID NO: 175 - DNA, 20 bp, Artificial Sequence
  Synthesized Oligonucleotide
ttacagagttaactgttacc

SEQ ID NO: 176 - DNA, 20 bp, Artificial Sequence
  Synthesized Oligonucleotide
atacaaatccagcacgctct

SEQ ID NO: 177 - DNA, 19 bp, Artificial Sequence
  Synthesized Oligonucleotide
tkggaccmacataacctaa

SEQ ID NO: 178 - DNA, 20 bp, Artificial Sequence
  Synthesized Oligonucleotide
ggacccacataacctaaaag

SEQ ID NO: 179 - DNA, 20 bp, Artificial Sequence
  Synthesized Oligonucleotide
ctaacaaaacacccaaagaa

SEQ ID NO: 180 - DNA, 23 bp, Artificial Sequence
  Synthesized Oligonucleotide
cctgaatctgctaataatatttc

SEQ ID NO: 181 - DNA, 20 bp, Artificial Sequence
  Synthesized Oligonucleotide
tcaccaggttcaacccaaaa

SEQ ID NO: 182 - DNA, 21 bp, Artificial Sequence
  Synthesized Oligonucleotide
ggccatatcctgaatctgcta

SEQ ID NO: 183 - DNA, 23 bp, Artificial Sequence
  Synthesized Oligonucleotide
gcaagcaatagaatcatcagaca

SEQ ID NO: 184 - DNA, 27 bp, Artificial Sequence
  Synthesized Oligonucleotide
ttggtacaaatgaatctggctgaaccc

SEQ ID NO: 185 - DNA, 26 bp, Artificial Sequence
  Synthesized Oligonucleotide
tgcttcgttcaatggataaacacggc

SEQ ID NO: 186 - DNA, 26 bp, Artificial Sequence
  Synthesized Oligonucleotide
tggctcctaatgctaatgcaatgcgg

SEQ ID NO: 187 - DNA, 27 bp, Artificial Sequence
  Synthesized Oligonucleotide
ccgcattgcattagcattaggagccaa

(19) United States
(12) Patent Application Publication
(10) Pub. No.: US 2015/0232919 A1
(43) Pub. Date: Aug. 20, 2015

(54) SEQUENCES FOR DETECTION AND IDENTIFICATION OF METHICILLIN-RESISTANT STAPHYLOCOCCUS AUREUS (MRSA) OF MREJ TYPE XXI

(71) Applicant: GeneOhm Sciences Canada, Inc., Sainte-Foy (CA)

(72) Inventors: Christian Menard, St-Lambert-de-Lauzon (CA); Celine Roger-Dalbert, Borel (CA)

(21) Appl. No.: 14/390,752

(22) PCT Filed: Mar. 14, 2013
(86) PCT No.: PCT/IB2013/000900

§ 371 Date: Oct. 3, 2014

Related U.S. Application Data
(60) Provisional application No. 61/621,368, filed on Apr. 6, 2012.

(51) Int. Cl. C12Q 1/68

(52) U.S. Cl. CPC C12Q 1/689; C12Q 2600/156

(57) ABSTRACT

Provided herein are compositions and methods for the detection and identification of Staphylococcus aureus strains harboring polymorphic SCCmec right extremity (MREP) type xxi sequences.

### REFERENCE TO SEQUENCE LISTING, TABLE, OR COMPUTER PROGRAM LISTING

The present application is being filed along with a Sequence Listing in electronic format. The Sequence Listing is provided as a file entitled GENOM.114A.txt, last saved Mar. 14, 2013, which is 85.6 kb in size. The information is incorporated herein by reference in its entirety.

### BACKGROUND OF THE INVENTION

1. Field

The embodiments disclosed herein relate to molecular diagnostic tools for the detection of methicillin-resistant Staphylococcus aureus.

2. Related Art

The coagulase-positive species Staphylococcus aureus (S. aureus) is well documented as a human opportunistic pathogen (Murray et al. Eds, 1999, Manual of Clinical Microbiology, 7th ed., ASM Press, Washington, D.C.). Nosocomial infections caused by S. aureus are a major cause of morbidity and mortality. Some of the most common infections caused by S. aureus involve the skin, and they include furuncles or boils, cellulitis, impetigo, and postoperative wound infections at various sites. Some of the more serious infections produced by S. aureus are bacteremia, pneumonia, osteomyelitis, acute endocarditis, myocarditis, pericarditis, cerebritis, meningitis, scalded skin syndrome, and various abscesses. Food poisoning mediated by staphylococcal enterotoxins is another important syndrome associated with S. aureus. Toxic shock syndrome, a community-acquired disease, has also been attributed to infection or colonization with toxigenic S. aureus.

Methicillin-resistant S. aureus (MRSA) emerged in the 1980s as a major clinical and epidemiologic problem in hospitals (Oliveira et al., (2002) Lancet Infect Dis. 2:180-9). MRSA are resistant to all β-lactams including penicillins, cephalosporins, carbapenems, and monobactams, which are the most commonly used antibiotics to cure S. aureus infections.

MRSA infections can only be treated with toxic and more costly antibiotics, which are normally used as the last line of defense. Since MRSA can spread easily from patient to patient via personnel, hospitals over the world are confronted with the problem of controlling MRSA.

Not that long ago, the only way to know a strain's resistance was to perform a manual antimicrobial susceptibility testing. Antimicrobial susceptibility testing suffers from many drawbacks, including the extensive time (at least 48 hours) before the results are available, a lack of reproducibility, a lack of standardization of the process, and user errors. Consequently, there is a need to develop rapid and simple screening or diagnostic tests for detection and/or identification of MRSA to reduce its dissemination and improve the diagnosis and treatment of infected patients.

There is a need for compositions and methods for quick and sensitive detection of MRSA.

### SUMMARY

The embodiments disclosed herein are based, in part, upon the discovery that certain strains of Staphylococcus aureus, including those that harbor a mecA homolog gene, mecA_{LGA251}, share the same sequence located at the right extremity of the SCCmec region of the MRSA nucleic acids, i.e., the polymorphic right extremity junction. Provided herein are methods and compositions that can be used to detect these MRSA strains, which were heretofore undetectable by conventional commercial molecular based assays. Also provided herein are compositions and methods that allow for the further (e.g., either simultaneous or sequential) detection of Staphylococcus aureus generally, and/or for the further detection of mecA and/or mecA_{LGA251}, in addition to MRSA strains.

Accordingly, provided herein are methods and compositions for the detection of MRSA that harbor an MREJ type xxi sequence. Some embodiments provide a composition for the detection of methicillin-resistant Staphylococcus aureus (MRSA) having MREJ type xxi nucleic acids. The MRSA can include a Staphylococcal cassette chromosome mec (SCCmec) element including a mecA homolog (mecA_{LGA251}, or mecC), wherein the SCCmec cassette is inserted into S. aureus chromosomal DNA, thereby generating a polymorphic right extremity junction (MREJ) type xxi sequence that comprises polymorphic sequences from the right extremity and chromosomal DNA adjoining the polymorphic right extremity. The composition can include a first amplification primer that is between 10 and 45 nucleotides in length, and that specifically hybridizes under standard PCR conditions to the polymorphic right extremity sequences of the MREJ type xxi nucleic acids. In some embodiments, the first amplification primer specifically hybridizes to the nucleic acid sequence of SEQ ID NO:1 or the complement thereof under said standard PCR conditions.

The compositions disclosed herein can further include a second amplification primer between 10 and 45 nucleotides in length that specifically hybridizes under standard PCR conditions to S. aureus chromosomal sequences located within 1 kilobase from the insertion point of the SCCmec element into the chromosomal DNA. Preferably, the first and second amplification primers together generate an amplicon of the right extremity junction of MREJ type xxi nucleic acids under the standard PCR conditions in the presence of MRSA comprising MREJ type xxi nucleic acids. Accordingly, in some embodiments, the second amplification primer specifically hybridizes under standard PCR conditions to orfX. The compositions disclosed herein can further include a probe, e.g., an oligonucleotide probe comprises a fluorescence emitter moiety and a fluorescence quencher moiety, that specifically hybridizes to the amplicon of the MREJ type xxi nucleic acids under the standard PCR conditions.

The compositions disclosed herein can include one or more additional amplification primers between 10 and 45 nucleotides in length that specifically hybridize to one or more polymorphic SCCmec right extremity sequences from an MREJ type i to xx MRSA, i.e., to one or more polymorphic SCCmec right extremity sequences selected from the group consisting of SEQ ID NOs: 5 to 29.

The methods and compositions can also include further oligonucleotides, i.e., that are configured to specifically amplify mecA and/or mecA_{LGA251}/mecC sequences, and/or that are configured to specifically amplify Staphylococcus aureus-specific sequences.

Accordingly, some embodiments provide compositions wherein the first amplification primer is at least 80% identical to SEQ ID NO:2. In some embodiments, the compositions can include a second amplification primer that is at least 80% identical to SEQ ID NO:3. In some embodiments, the composition can include a probe, wherein the probe is at least 80% identical to SEQ ID NO:4 or 82.

In some embodiments, the compositions disclosed herein are provided in dried form, e.g., lyophilized or the like.

Also provided herein are methods for the detection of methicillin-resistant Staphylococcus aureus (MRSA) comprising MREJ type xxi nucleic acids, and for the detection of MREJ type xxi nucleic acids, wherein the S. aureus includes a Staphylococcal cassette chromosome mec (SCCmec) element including a mecA homolog (mecA_{LGA251} or mecC). The SCCmec cassette can be inserted into S. aureus chromosomal DNA, thereby generating a polymorphic right extremity junction (MREJ) type xxi sequence that comprises polymorphic sequences from the right extremity (MREP sequences) and chromosomal DNA adjoining the polymorphic right extremity. Accordingly, in some embodiments, the methods can include the steps of providing a test sample; contacting the sample with a first amplification primer between 10 and 45 nucleotides in length, that specifically hybridizes under standard PCR conditions to the polymorphic right extremity sequences of the MREJ type xxi sequence; wherein the contacting is performed under conditions wherein an amplicon of the mec right extremity junction of the MREJ type xxi nucleic acids is generated if S. aureus comprising MREJ type xxi nucleic acids is present in the sample. The method can also include the step of determining whether or not an amplicon of the MREJ type xxi nucleic acids is generated following the contacting step. In some embodiments, the first amplification primer specifically hybridizes to the nucleic acid sequence of SEQ ID NO:1 under said standard PCR conditions.

In some embodiments, the method can include contacting the sample with a second amplification primer between 10 and 45 nucleotides in length that hybridizes under the standard PCR conditions to the orfX gene of S. aureus, wherein said first and second amplification primer together generate an amplicon of the SCCmec right extremity junction (MREJ) region sequence of the SCCmec right extremity junction of MRSA under the standard PCR conditions in the presence MREJ type xxi nucleic acids.

The method of claim 15, further comprising contacting the sample with a probe, wherein said probe specifically hybridizes to the amplicon of the SCCmec right extremity junction (MREJ) region sequence of MREJ type xxi nucleic acids under the standard PCR conditions. In some embodiments, the probe includes a fluorescence emitter moiety and a fluorescence quencher moiety.

In some embodiments, the contacting step also includes contacting the sample with one or more additional amplification primers, wherein said one or more additional amplification primers are between 10 and 45 nucleotides in length that specifically hybridizes to one or polymorphic SCCmec right extremity sequence from an MREJ type i to xx MRSA. In some embodiments, the contacting step also includes contacting the sample with one or more additional amplification primers between 10 and 45 nucleotides in length that specifically hybridizes to and are configured to generate an amplicon of mecA sequences, mecC sequences and/or Staphylococcus aureus-specific sequences. Non-limiting examples of S. aureus-specific sequences include, but are not limited to nuc sequences, rRNA sequences, femB sequences, Sa442 sequences, and the like. The skilled artisan will readily appreciate, however, that any sequence that is unique to Staphylococcus aureus can be used in the embodiments described herein.

Accordingly, the contacting step of the methods described herein can include performing a nucleic acid amplification reaction, such as PCR, strand displacement amplification (SDA), for example multiple displacement amplification (MDA), loop-mediated isothermal amplification (LAMP), ligase chain reaction (LCR), immuno-amplification, nucleic acid sequence based amplification (NASBA), self-sustained sequence replication (3SR), or rolling circle amplification. In some preferred embodiments, the method includes performing multiplex PCR. In some preferred embodiments, the method includes performing real-time PCR.

### BRIEF DESCRIPTION OF THE DRAWINGS

FIG. 1 shows the sequence of a type xxi MREJ region. Also shown are the locations of various primers and probes disclosed in the embodiments described herein.

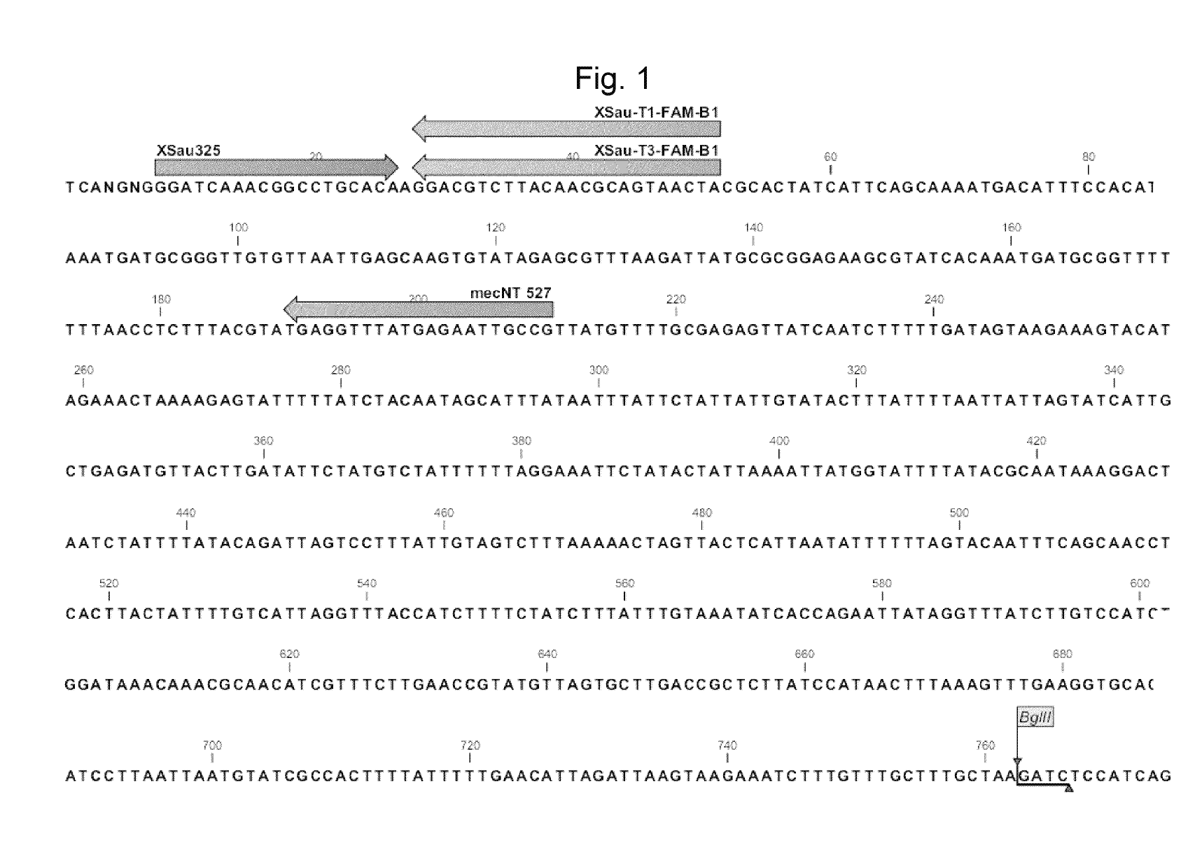

### DETAILED DESCRIPTION

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Provided herein are compositions and methods for the improved detection of methicillin-resistant Staphylococcus aureus (MRSA), and in particular, methods of detecting S. aureus harboring a mecA homolog gene, such as a CC130 or cc130 S. aureus strain. Methicillin resistance in Staphylococcus aureus is due to the gene product of the mecA gene, encoding for the penicillin binding protein 2a (PBP-2a), a β-lactam-resistant transpeptidase. mecA is absent from methicillin-sensitive S. aureus, but is widely distributed among other species of staphylococci, including coagulase negative staphylococci, (CoNS), such as Staphylococcus epidermidis, Staphylococcus haemolyticus, Staphylococcus capitis, S. saprophyticus S. lentus, S. hominus, S. cohnii, S. delphini, S. xylosus, S. muscae, S. schleiferi, S. coagulans, and others. The mecA gene is highly conserved (Ubukata et al., 1990, Antimicrob. Agents Chemother. 34:170-172). In methicillin-resistant staphylococci, mecA is present in a genetic element termed staphylococcal cassette chromosome mec (SCCmec), which is inserted into the chromosome of staphylococci.

SCCmec cassettes range from 20 kb to more than 60 kb in length, and include site specific recombinase genes and transposable elements, in addition to the mecA gene. SCCmec cassettes are inserted at a fixed location, termed “attBscc” within the chromosomal DNA of Staphylococcus aureus, and which is located at the 3′ end of an open reading frame termed “orfX.” Huletsky et al. (2004) J. Clin. Microbiol. 42(5):1875-1884. MRSA strains have been classified based upon the organization of the SCCmec cassettes (termed “SCCmec typing). Different SCCmecs have been classified according to their recombinase genes, and the genetic organization of mecI and mecR genes, which are regulators of mecA.

Many molecular assays for the detection and identification of MRSA involve the detection of the mecA gene. Since mecA is also found in CoNS, however, detection of mecA alone is not sufficient to determine the presence of MRSA, as methicillin-resistant CoNS will also test positive. In order to address this problem, assays have been developed in which S. aureus-specific genes are detected, in addition to mecA. See, Schuenck et al., Res. Microbiol., (2006), in press, Shittu et al., (2006), Diagn Microbiol Infect Dis. July 17, Grisold et al., (2006), Methods Mol. Biol. 345: 79-89, Costa et al., (2005), Diag. Microbiol. and Infect. Dis, 51: 13-17, Mc Donald et al., (2005), J. Clin. Microbiol., 43: 6147-6149, Zhang et al., (2005), J. Clin. Microbiol. 43: 5026-5033, Hagen et al. (2005), Int J Med Microbiol. 295:77-86, Maes, et al. (2002) J. Clin. Microbiol. 40:1514-1517, Saito et al., (1995) J. Clin. Microbiol. 33:2498-2500; Ubukata et al., (1992) J. Clin. Microbiol. 30:1728-1733; Murakami et al., (1991) J. Clin. Microbiol. 29:2240-2244; Hiramatsu et al., (1992) Microbiol. Immunol. 36:445-453). Furthermore, Levi and Towner (2003), J. Clin. Microbiol., 41:3890-3892 and Poulsen et al. (2003), J Antimicrob Chemother., 51:419-421 describe detection of methicillin resistance in coagulase-negative Staphylococci and in S. aureus using the EVIGENE™ MRSA Detection kit.

However, because the mecA gene is widely distributed in both S. aureus and coagulase-negative staphylococci, each of the methods described above are incapable of discriminating between samples containing both methicillin-sensitive S. aureus (“MSSA”) and methicillin-resistant coagulase-negative staphylococci, and samples that contain only MRSA or that have both methicillin-sensitive S. aureus and MRSA.

To address this problem, Hiramatsu et al. developed a PCR-based assay specific for MRSA that utilizes primers that hybridize to the right extremities of DNA of SCCmec types I-III in combination with primers specific to the S. aureus chromosome, which corresponds to the nucleotide sequence on the right side of the SCCmec integration site. (U.S. Pat. No. 6,156,507, hereinafter the “'507 patent”). More recently, Zhang et al., (2005), J. Clin. Microbiol. 43: 5026-5033, described a multiplex assay for subtyping SCCmec types I to V MRSA. Nucleotide sequences surrounding the SCCmec integration site in other staphylococcal species (e.g., S. epidermidis and S. haemolyticus) are different from those found in S. aureus, therefore multiplex PCR assays that utilize oligonucleotides that hybridize to the right extremities of SCCmec and the S. aureus chromosome have the advantage of being specific for the detection of MRSA.

The PCR assay described in the '507 patent also led to the development of “MREP typing” (mec right extremity polymorphism) of SCCmec DNA (Ito et al., (2001) Antimicrob. Agents Chemother. 45:1323-1336; Hiramatsu et al., (1996) J. Infect. Chemother. 2:117-129). The MREP typing method takes advantage of the fact that the nucleotide sequences of the three MREP types differ at the right extremity of SCCmec DNAs adjacent to the integration site among the three types of SCCmec.

The term “MREJ” refers to the mec right extremity junction <<mec right extremity junction>>. MREJ region nucleic acid sequences are approximately 1 kilobase (kb) in length and include sequences from the SCCmec right extremity as well as bacterial chromosomal DNA to the right of the SCCmec integration site. Strains that were classified as MREP types i-iii correspond to MREJ types i-iii. MREJ types iv to xx have been previously characterized Huletsky et al., (2004), J. Clin. Microbiol. 42:1875-1884; International Patent Application PCT/CA02/00824, United States Patent Application 2008/0227087.

Recently, Garcia-Alvarez et al. reported on bacterial strains that were confirmed by ribosomal RNA analysis to be S. aureus, and which exhibited resistance to methicillin using routine antibiotic susceptibility testing. Garcia-Alvarez et al. (2011) Lancet 11:595-603. Surprisingly, however, these strains tested negative as MRSA using the assays described above, which rely upon the detection of mecA or the detection of known MREJ regions. Assay specificity is limited to identify MREJ regions. Garcia-Alvarez et al. demonstrated that the strains harbored a novel mecA homolog that shared limited homology with known mecA genes, i.e., 63% identity at the amino acid level and 70% identity at the DNA level, explaining the inability of the assays relying upon the detection of the mecA gene to detect these MRSA. The mecA homolog was termed mecA_{LGA251}, also known herein as mecC. As the assays utilizing MREJ amplification were also unable to detect any of the mecA_{LGA251} MRSA strains, mecA_{LGA251} MRSA strains would be incorrectly identified as false negative. This error in diagnosing and identifying MRSA could have serious impact on the patient health outcome.

Several strains of S. aureus that tested positive as methicillin resistant using standard antibiotic susceptibility testing, yet that were not detected as MRSA using conventional, commercial molecular assays were analyzed further. The strains are listed in Table 1, below.

TABLE 1

| Staphylococcus aureus | |
|---|---|
| strain designation | Country of Origin |
| IDI-6112 | France |
| IDI-6113 | France |
| IDI-6121 | United Kingdom |
| IDI-6122 | United Kingdom |
| IDI-6123 | United Kingdom |
| IDI-6125 | United Kingdom |
| IDI-6126 | United Kingdom |
| IDI-6127 | United Kingdom |
| IDI-6128 | United Kingdom |
| IDI-6129 | United Kingdom |
| IDI-6130 | United Kingdom |
| IDI-6131 | United Kingdom |
| IDI-6132 | United Kingdom |
| IDI-6133 | United Kingdom |
| IDI-6134 | United Kingdom |
| IDI-6135 | United Kingdom |
| IDI-6136 | United Kingdom |
| IDI-6137 | United Kingdom |
| IDI-6138 | United Kingdom |
| IDI-6139 | United Kingdom |
| IDI-6140 | United Kingdom |
| IDI-6141 | United Kingdom |
| IDI-6142 | United Kingdom |
| IDI-6143 | United Kingdom |
| IDI-6144 | United Kingdom |
| IDI-6145 | United Kingdom |
| IDI-6146 | United Kingdom |
| IDI-6147 | Denmark |
| IDI-6148 | Denmark |
| IDI-6149 | Denmark |
| IDI-6150 | Denmark |
| IDI-6151 | Denmark |
| IDI-6152 | Denmark |
| IDI-6153 | Denmark |
| IDI-6154 | Denmark |
| IDI-6155 | Denmark |
| IDI-6156 | Denmark |
| IDI-6157 | Denmark |
| IDI-6158 | Denmark |
| IDI-6159 | Denmark |
| IDI-6160 | Denmark |
| IDI-6161 | Denmark |
| IDI-6162 | Denmark |
| IDI-6163 | Denmark |
| IDI-6164 | Denmark |
| IDI-6165 | Denmark |
| IDI-6166 | Denmark |
| IDI-6167 | Denmark |
| IDI-6168 | France |
| IDI-6170 | France |
| IDI-6171 | France |
| IDI-6208 | Germany |
| IDI-6209 | Germany |
| IDI-6211 | Germany |
| IDI-6213 | Germany |
| IDI-6214 | Germany |
| IDI-6215 | Germany |
| IDI-6216 | Germany |
| IDI-6217 | Germany |
| IDI-6218 | Germany |
| IDI-6219 | Germany |
| IDI-6220 | Germany |
| IDI-6221 | Germany |

As described herein, the present disclosure is based, in part, upon the surprising finding that the vast majority of MRSA strains analyzed that harbor the mecA homolog gene, mecA_{LGA251}/mecC, share the same MREJ sequence. Based upon this surprising finding, compositions and methods for the improved detection of MRSA are provided herein. The compositions and methods disclosed herein advantageously allow for reliable and rapid detection and identification of mecA_{LGA251} MRSA strains.

### Oligonucleotides

According to some embodiments disclosed herein, oligonucleotides are provided, for example amplification primers and/or sequence-specific probes. As used herein, the terms “primer” and “probe” include, but are not limited to oligonucleotides. Preferably, the oligonucleotide primers and/or probes disclosed herein can be between 8 and 45 nucleotides in length. For example, the primers and or probes can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers and/or probes can be provided in any suitable form, included bound to a solid support, liquid, and lyophilized, for example. The primer and probe sequences disclosed herein can be modified to contain additional nucleotides at the 5′ or the 3′ terminus, or both. The skilled artisan will appreciate, however, that additional bases to the 3′ terminus of amplification primers (not necessarily probes) are generally complementary to the template sequence. The primer and probe sequences disclosed herein can also be modified to remove nucleotides at the 5′ or the 3′ terminus. The skilled artisan will appreciate that in order to function for amplification, the primers or probes will be of a minimum length and annealing temperature as disclosed herein.

Oligonucleotide primers and probes can bind to their targets at an annealing temperature, which is a temperature less than the melting temperature (Tₘ). As used herein, “Tₘ” and “melting temperature” are interchangeable terms which refer to the temperature at which 50% of a population of double-stranded polynucleotide molecules becomes dissociated into single strands. Formulae for calculating the Tₘ of polynucleotides are well known in the art. For example, the Tₘ may be calculated by the following equation: Tₘ=69.3+0.41×(G+C)%−6−50/L, wherein L is the length of the probe in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.]. See, e.g., C. R. Newton et al. PCR, 2nd ed., Springer-Verlag (New York: 1997), p. 24. Other more sophisticated computations exist in the art, which take structural as well as sequence characteristics into account for the calculation of Tₘ. The melting temperature of an oligonucleotide can depend on complementarity between the oligonucleotide primer or probe and the binding sequence, and on salt conditions. In some embodiments, an oligonucleotide primer or probe provided herein has a Tₘ of less than about 90° C. in 50 mM KCl, 10 mM Tris-HCl buffer, for example about 89° C., 88, 87, 86, 85, 84, 83, 82, 81, 80 79, 78, 77, 76, 75, 74, 73, 72, 71, 70, 69, 68, 67, 66, 65, 64, 63, 62, 61, 60, 59, 58, 57, 56, 55, 54, 53, 52, 50, 49, 48, 47, 46, 45, 44, 43, 42, 41, 40, 39° C., or less, including ranges between any two of the listed values.

As discussed in further detail below, in some embodiments, the primers disclosed herein, e.g., amplification primers, can be provided as an amplification primer pair, e.g., comprising a forward primer and a reverse primer (first amplification primer and second amplification primer). Preferably, the forward and reverse primers have Tₘ's that do not differ by more than 10° C., e.g., that differ by less than 10° C., less than 9° C., less than 8° C., less than 7° C., less than 6° C., less than 5° C., less than 4° C., less than 3° C., less than 2° C., or less than 1° C.

The primer and probe sequences may be modified by having nucleotide substitutions (relative to the target sequence) within the oligonucleotide sequence, provided that the oligonucleotide contains enough complementarity to hybridize specifically to the target nucleic acid sequence. In this manner, at least 1, 2, 3, 4, or up to about 5 nucleotides can be substituted. As used herein, the term “complementary” refers to sequence complementarity between regions of two polynucleotide strands or between two regions of the same polynucleotide strand. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Fully complementary” refers to a first polynucleotide that is 100% or “fully” complementary to a second polynucleotide and thus forms a base pair at every nucleotide position. “Partially complementary” also refers to a first polynucleotide that is not 100% complementary (e.g., 90%, or 80% or 70% complementary) and contains mismatched nucleotides at one or more nucleotide positions. In some embodiments, an oligonucleotide includes a universal base.

As used herein, the term “hybridization” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature of the formed hybrid, the presence of other components (e.g., the presence or absence of polyethylene glycol), the molarity of the hybridizing strands and the G:C content of the polynucleotide strands. In some embodiments, e.g., embodiments providing more than one oligonucleotide, the oligonucleotides are designed such that the Tₘ of one oligonucleotide is within 2° C. of the Tₘ of the other oligonucleotide. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al, eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). As discussed further herein, the term “specific hybridization” or “specifically hybridizes” refers to the hybridization of a polynucleotide, e.g., an oligonucleotide primer or probe or the like to a target sequence, such as a sequence to be quantified in a sample, a positive control target nucleic acid sequence, or the like, and not to unrelated sequences, under conditions typically used for nucleic acid amplification.

In some embodiments, the primers and/or probes include oligonucleotides that hybridize to a target nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard nucleic acid amplification conditions as discussed below. As used herein, the term “substantially complementary” refers to the complementarity between two nucleic acids, e.g., the complementary region of the oligonucleotide and the target sequence. The complementarity need not be perfect; there may be any number of base pair mismatches that between the two nucleic acids. However, if the number of mismatches is so great that no hybridization can occur under even the least stringent of hybridization conditions, the sequence is not a substantially complementary sequence. When two sequences are referred to as “substantially complementary” herein, it is meant that the sequences are sufficiently complementary to the each other to hybridize under the selected reaction conditions. The relationship of nucleic acid complementarity and stringency of hybridization sufficient to achieve specificity is well known in the art and described further below in reference to sequence identity, melting temperature and hybridization conditions. Therefore, substantially complementary sequences can be used in any of the detection methods disclosed herein. Such probes can be, for example, perfectly complementary or can contain from 1 to many mismatches so long as the hybridization conditions are sufficient to allow, for example discrimination between a target sequence and a non-target sequence. Accordingly, substantially complementary sequences can refer to sequences ranging in percent identity from 100, 99, 98, 97, 96, 95, 94, 93, 92, 91, 90, 89, 88, 87, 86, 85, 84, 83, 82, 81, 80, 75, 70 or less, or any number in between, compared to the reference sequence. For example, the oligonucleotides disclosed herein can contain 1, 2, 3, 4, 5, or more mismatches and/or degenerate bases, as compared to the target sequence to which the oligonucleotide hybridizes, with the proviso that the oligonucleotides are capable of specifically hybridizing to the target sequence under, for example, standard nucleic acid amplification conditions.

Accordingly, by way of example, the term “stringent hybridization conditions” can refer to either or both of the following: a) 6×SSC at about 45° C., followed by one or more washes in 0.2×SSC, 0.1% SDS at 65° C., and b) 400 mM NaCl, 40 mM PIPES pH 6.4, 1 mM EDTA, 50° C. or 70° C. for 12-16 hours, followed by washing. In some embodiments, the term “stringent conditions” can refer to standard nucleic acid amplification (e.g., PCR) conditions.

In some embodiments, the sample or specimen is contacted with a set of amplification primers under standard nucleic acid amplification conditions, which are discussed in further detail below. For a review of PCR technology, including standard nucleic acid amplification conditions such as PCR conditions, applied to clinical microbiology, see DNA Methods in Clinical Microbiology, Singleton P., published by Dordrecht; Boston: Kluwer Academic, (2000) Molecular Cloning to Genetic Engineering White, B. A. Ed. in Methods in Molecular Biology 67: Humana Press, Totowa (1997) and “PCR Methods and Applications”, from 1991 to 1995 (Cold Spring Harbor Laboratory Press). Non-limiting examples of “nucleic acid amplification conditions” and “PCR conditions” include the conditions disclosed in the references cited herein, such as, for example, 50 mM KCl, 10 mM Tris-HCl (pH 9.0), 0.1% Triton X-100, 2.5 mM MgCl₂, with an annealing temperature of 72° C.; or 4 mM MgCl₂, 100 mM Tris, pH 8.3, 10 mM KCl, 5 mM (NH₄)₂SO₄, 0.15 mg BSA, 4% Trehalose, with an annealing temperature of 59° C., or 50 mM KCl, 10 mM Tris-HCl (pH 9.0), 0.1% Triton X-100, 2.5 mM MgCl₂, with an annealing temperature of 55° C., or the like.

The primers described herein can be prepared using techniques known in the art, including, but not limited to, cloning and digestion of the appropriate sequences and direct chemical synthesis. Chemical synthesis methods that can be used to make the primers of the described herein, include, but are not limited to, the phosphotriester method described by Narang et al. (1979) Methods in Enzymology 68:90, the phosphodiester method disclosed by Brown et al. (1979) Methods in Enzymology 68:109, the diethylphosphoramidate method disclosed by Beaucage et al. (1981) Tetrahedron Letters 22:1859, and the solid support method described in U.S. Pat. No. 4,458,066. The use of an automated oligonucleotide synthesizer to prepare synthetic oligonucleotide primers described herein is also contemplated herein.

Accordingly, some embodiments relate to compositions that comprise oligonucleotides (e.g., an amplification primers and probes) that specifically hybridize (e.g., under standard nucleic acid amplification conditions, e.g., standard PCR conditions, and/or stringent hybridization conditions) to the polymorphic SCCmec right extremity sequences in MRSA strains that have MREJ type xxi sequences. For example, in some embodiments, provided are compositions that comprise oligonucleotides that specifically hybridize to the polymorphic SCCmec right extremity sequences present in SEQ ID NO: 1, or the complement thereof e.g., within nucleotide positions 1 to 164 of SEQ ID NO:1, or its complement. An exemplary oligonucleotide that specifically hybridizes to the polymorphic SCCmec right extremity sequences of MREJ type xxi, including the polymorphic right extremity sequences within SEQ ID NO: 1, is provided in SEQ ID NO:2, or the complement thereof. Thus, provided herein are oligonucleotides that are substantially complementary to SEQ ID NO:2 or the complement thereof, as well as oligonucleotides containing 1, 2, 3, 4 or more mismatches or universal nucleotides relative to SEQ ID NO:2 or the complement thereof, e.g., including oligonucleotides that are at least 80% identical (for example at least 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 2 or the complement thereof.

In some embodiments, the compositions and methods can include oligonucleotides, e.g., amplification primers or sequence-specific probes, that specifically hybridize to one or more polymorphic right extremity sequences within MREJ regions other than MREJ type xxi. Accordingly, some embodiments provide oligonucleotides, e.g., amplification primers or sequence-specific probes that specifically hybridize (under standard nucleic acid amplification conditions, and/or stringent hybridization conditions) to polymorphic right extremity sequences within one or more MREJ regions selected from MREJ type i regions, MREJ type ii regions, MREJ type iii regions, MREJ type iv regions, MREJ type v regions, MREJ type vi regions, MREJ type vii regions, MREJ type viii regions, MREJ type ix regions, MREJ type x regions, MREJ type xi regions, MREJ type xii regions, MREJ type xiii regions, MREJ type xiv regions, MREJ type xv regions, MREJ type xvi regions, MREJ type xvii regions, MREJ type xviii regions, MREJ type xix regions, and MREJ type xx regions.

In some embodiments, the compositions and methods can include oligonucleotides, e.g., amplification primers that specifically hybridize to, and are capable of generating an amplicon of, mecA sequences, or a fragment thereof. Accordingly, some embodiments include oligonucleotides, e.g., amplification primers that specifically hybridize to and are capable of generating an amplicon of sequences within SEQ ID NO:156. Some embodiments include oligonucleotides, e.g., amplification primers that specifically hybridize to, and are capable of generating an amplicon of, mecC sequences, e.g., sequences within SEQ ID NO:157, or a fragment thereof. Some embodiments include oligonucleotides, e.g., amplification primers that specifically hybridize to and are capable of generating an amplicon of Staphylococcus aureus-specific sequences. For example, some embodiments provide oligonucleotides that specifically hybridize to and are capable of generating an amplicon of nuc sequences (e.g., sequences derived from SEQ ID NO:158), femB sequences (e.g., sequences derived from SEQ ID NO: 159), Sa442 sequences (e.g., from Martineau, et al. 1998, J. Clin. Microbiol. 36(3):618-623) (SEQ ID NO:160).

In some embodiments, provided is an oligonucleotide that specifically hybridizes to the polymorphic right extremity sequences of an MREJ type xxi region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type i region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type ii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type iii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type iv region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type v region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type vi region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type vii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type viii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type ix region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type x region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xi region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xiii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xiv region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xv region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xvi region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xvii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xviii region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xix region under standard conditions for nucleic acid amplification, and/or stringent hybridization conditions. In some embodiments, the sequence specific oligonucleotides (e.g. amplification primers and sequence specific probes) disclosed herein specifically hybridize to the polymorphic right extremity sequences of an MREJ type xx region under standard conditions for nucleic acid amplification, and/or stringent hybridization conditions. In some embodiments, the sequence specific oligonucleotides (e.g., amplification primers and/or sequence specific probes) disclosed herein specifically hybridize to mecA sequences. In some embodiments, the sequence specific oligonucleotides (e.g., amplification primers and/or sequence specific probes) disclosed herein specifically hybridize to mecC sequences. In some embodiments, the sequence specific oligonucleotides (e.g., amplification primers and/or sequence specific probes) disclosed herein specifically hybridize to nuc sequences. In some embodiments, the sequence specific oligonucleotides (e.g., amplification primers and/or sequence specific probes) disclosed herein specifically hybridize to Sa442 sequences. In some embodiments, the sequence specific oligonucleotides (e.g., amplification primers and/or sequence specific probes) disclosed herein specifically hybridize to femB sequences.

Exemplary MREJ region sequences related to the embodiments disclosed herein include, for example:

| MREJ type | Exemplified in SEQ ID NO(s): |
|---|---|
| i | 5 |
| ii | 6 |
| iii | 7 |
| iv | 8 |
| v | 9 |
| vi | 10 |
| vii | 11 |
| viii | 12 |
| ix | 13 |
| x | 14 |
| xi | 15, 16 and 17 |
| xii | 18 |
| xiii | 19, 20, 21 |
| xiv | 22 |
| xv | 23 |
| xvi | 24 |
| xvii | 25 |
| xviii | 26 and 27 |
| xix | 28 |
| xx | 29 |
| xxi | 1 |

In addition to the oligonucleotide of SEQ ID NO:2, discussed above, which specifically hybridizes to the polymorphic right extremity sequences within MREJ type xxi regions, oligonucleotides that are specific for one or more polymorphic sequences within MREJ region sequences, or for S. aureus chromosomal DNA sequences, useful in the embodiments disclosed herein include, but are not limited to the following:

| Specific for: | SEQ ID NO |
|---|---|
| MREJ type xi primer | 30 |
| MREJ type xi primer | 31 |
| MREJ type xii primer | 32 |
| MREJ type xii primer | 33 |
| MREJ type ix, xiii, xiv primer | 34 |
| MREJ type xv primer | 35 |
| MREJ type xv primer | 36 |
| MREJ type xv primer | 37 |
| MREJ type xv primer | 38 |
| MREJ type i, ii and xvi primer | 39 |
| MREJ type xvii primer | 40 |
| MREJ type xvii primer | 41 |
| MREJ type xvii primer | 42 |
| MREJ type xviii primer | 43 |
| MREJ type xix primer | 44 |
| MREJ type xx primer | 45 |
| orfX | 46 |
| orfX r | 47 |
| orfX | 49 |
| orfX | 50 |
| orfX | 51 |
| orfX | 52 |
| orfX | 53 |
| orfX | 54 |
| orfX | 55 |
| orfX | 56 |
| orfX | 57 |
| orfX | 58 |
| orfX | 59 |
| orfX | 60 |
| orfX | 61 |
| orfX | 62 |
| orfX | 63 |
| orfX | 64 |
| MREJ types i and ii | 65 |
| MREJ types i and ii | 66 |
| MREJ types i and ii | 67 |
| MREJ type ii | 68 |
| MREJ type ii | 69 |
| MREJ type iii | 70 |
| MREJ type iii | 71 |
| MREJ type iii | 72 |
| MREJ type iv | 73 |
| MREJ type v | 74 |
| MREJ type vi | 75 |
| MREJ type vi | 76 |
| MREJ type vii | 77 |
| MREJ type vii | 78 |
| MREJ type viii | 79 |
| MREJ type viii | 80 |
| MREJ type ix | 81 |
| MREJ type x | 83 |
| nuc | 161 |
| nuc | 162 |
| nuc | 163 |
| nuc | 164 |
| nuc | 165 |
| nuc | 166 |
| nuc | 167 |
| nuc | 168 |
| nuc | 169 |
| nuc | 170 |
| nuc | 171 |
| nuc | 172 |
| Sa442 | 173 |
| Sa442 | 174 |
| femB | 175 |
| femB | 176 |
| mecA | 177 |
| mecA | 178 |
| mecA | 179 |
| mecC | 180 |
| mecC | 181 |
| mecC | 182 |
| mecC | 183 |
| mecA | 184 |
| mecA | 185 |
| mecA | 186 |
| mecC | 187 |

In addition to the foregoing, the skilled artisan will appreciate that the compositions and methods disclosed herein can include primers and/or probes for the specific detection of the right extremity region of the SCCmec sequences in various MRSA strains in addition to those mentioned herein above. By way of example, in some embodiments, the compositions and methods disclosed herein include sequences, e.g., primers and/or probes, described in International Patent Application Publication No. WO 08/080620, including variants thereof, and complements thereof. Accordingly, the compositions and methods disclosed herein can include primers and/or probes listed below:

TABLE 2

| SEQ | |
|---|---|
| ID NO: | Sequence (5′-3′) |
| 84 | GCA ATT CAC ATA AAC CTC ATA TGT TC |
| 85 | ACC TCA TAT GTT CTG ATA CAT TCA |
| 86 | GCA ATT CAC ATA AAC CTC ATA T |
| 87 | CAT AAC AGC AAT TCA CAT AAA CCT C |
| 88 | TAA CAG CAA TTC ACA TAA ACC T |
| 89 | CGC TAT TAT TTA CTT GAA ATG AAA GAC |
| 90 | CTT GAA ATG AAA GAC TGC GGA |
| 91 | TTG CTT CAC TAT AAG TAT TCA GTA TAA AGA ATT TAC TTG AAA TGA AAG ACT GCG |
| 92 | ATT TAC TTG AAA TGA AAG ACT GCG |
| 93 | AAA GAA TAT TTC GCT ATT ATT TAC TTG AA |
| 94 | TCA GTA TAA AGA ATA TTT CGC TAT TAT TT |
| 95 | TGA AAT GAA AGA CTG CGG AG |
| 96 | AAC CTC ATA TGT TCT GAT ACA TTC AAA |
| 97 | TAT GTC AAA AAT CAT GAA CCT CAT TAC T |
| 98 | CAT AAC AGC AAT TCA CAT AAA CCT C |
| 99 | GAC TGC GGA GGC TAA CT |
| 100 | ATC CCT TTA TGA AGC GGC |
| 101 | TGA AAT GAA AGA CTG CGG AG |
| 102 | GCA AGG TAT AAT CCA ATA TTT CAT ATA TGT |
| 103 | AGT TCC ATA ATC AAT ATA ATT TGT ACA GT |
| 104 | ACA TCG TAT GAT ATT GCA AGG TA |
| 105 | CTT TCA TTC TTT CTT GAT TCC ATT AG |
| 106 | CAC TCT ATA AAC ATC GTA TGA TAT TGC |
| 107 | TTC TTA ATT TAA TTG TAG TTC CAT AAT CAA |
| 108 | AAT TAT ACA CAA CCT AAT TTT TAG TTT TAT |
| 109 | AAT TTT TAG TTT TAT TTA TGA TAC GCT TC |
| 110 | ACA CAA CCT AAT TTT TAG TTT TAT TTA TGA |
| 111 | TTT ATT AAA CAC TCT ATA AAC ATC GTA TGA |
| 112 | TCA CAT CTC ATT AAA TTT TTA AAT TAT ACA C |
| 113 | CCA CAT CTC ATT AAA TTT TTA AAT TAT ACA C |
| 114 | ATA TTA TAC ACA ATC CGT TTT TTA GTT TTA |
| 115 | ACA CAA TCC GTT TTT TAG TTT TAT TTA TG |
| 116 | TTC TAA TTT ATT TAA CAT AAA ATC AAT CCT |
| 117 | CAA TCC TTT TTA TAT TTA AAA TAT ATT ATA CAC |
| 118 | AAG TCG CTT TGC CTT TGG GTC A |
| 119 | TAC AAA GTC GCT TTG CCT TTG GGT CA |
| 120 | GGC CGT TTG ATC CGC CAA T |
| 121 | AAG TCG CTT TGC CCT TGG GTA |
| 122 | AAG TCG CTT TGC CCT TGG GT |
| 123 | AAG TCG CTT TGC CCT TGG GTC A |
| 124 | AAG TCG CTT TGC CCT TGG G |
| 125 | CAA GAA TTG AAC CAA CGC AT |
| 126 | CAA TGA CGA ATA CAT AGT CGC TTT GCC CTT |
| 127 | CGT TTG ATC CGC CAA TGA CGA |
| 128 | GCC AAT CCT TCG GAA GAT AGC A |
| 129 | ATT AAC ACA ACC CGC ATC |
| 130 | GTC GCT TTG CCC TTG GGT C |
| 131 | TCG CTT TGC CCT TGG GTC AT |
| 132 | GGC CGT TTG ATC CGC CAA T |
| 133 | GTC CTT GTG CAG GCC GTT TGA T |
| 134 | CTT GGG TCA TGC GTT GGT TCA ATT |
| 135 | CGA ATA CAA AGT CGC TTT GCC CTT GGG |
| 136 | ATG CGT TGG TTC AAT TCT TG |
| 137 | GCG TTG GTT CAA TTC TTG GG |
| 138 | ACC CAA GGG CAA AGC GAC TT |
| 139 | GGT AAT GCG TTG GTT CAA TTC TTG |
| 140 | ACA AAG TCG CTA TGC CCT TGG GTC A |
| 141 | CTT TCC TTG TAT TTC TAA TGT AAT GAC TG |
| 142 | TTG ATG TGG GAA TGT CAT TTT GCT GAA |
| 143 | GCG TTG GTT CAA TTC TTG GGC CAA T |
| 144 | GTT GGT TCA ATT CTT GGG CCA ATC CTT CG |
| 145 | CGA ATA CAA AGT CGC TTT GCC CTT GG |
| 146 | GCC AAT GAC GAA TAC AAA GTC GCT TTG CC |
| 147 | TGG GCC AAT CCT TCG GAA GAT AGC A |
| 148 | ATG CGT TGG TTC GAT TCT TG |
| 149 | CAT GCG TTG GTT CGA TTC TTG |
| 150 | AAG TCG CTT TGC CCT TGG G |
| 151 | CAT GCG TTG GTT CGA TTC TTG |
| 152 | AAG TCG CTT TGC CCT TGG GTC AT |
| 153 | TGC TCA ATT AAC ACA ACC CGC ATC A |
| 154 | GCC GCG CTG CTC AAT TAA CAC AAC CCG CGC GGC |
| 155 | GCC GCG CAT GCG TTG GTT CAA TTC TGC GCG GC |

Accordingly, in exemplary embodiments, provided are methods and compositions for the detection of MRSA comprising MREJ xxi, and one or more MREJ types selected from the group consisting of MREJ type i-xx. For example, some embodiments provide for the detection and/or identification of MRSA comprising type i, ii, iii and xxi MREJ nucleic acids, using the MREJ-specific and S. aureus chromosomal DNA-specific oligonucleotides disclosed herein. Some embodiments provide for the detection and/or identification of MRSA comprising type i, ii, iii, iv and xxi MREJ sequences, using a combination of MREJ-specific oligonucleotides as described herein. Some embodiments provide for the detection of MRSA comprising type i, ii, iii, iv, v, vii, and xxi MREJ nucleic acids, using a combination of MREJ-specific oligonucleotides disclosed herein. Some embodiments provide for the identification of MRSA comprising type i, ii, iii, iv, v, vii, and xxi MREJ nucleic acids, using a combination of MREJ-specific oligonucleotides disclosed herein. Some embodiments provide for the detection of MRSA comprising type i, ii, iii, iv, v, vii, and xxi MREJ nucleic acids, using a combination of MREJ-specific oligonucleotides disclosed herein. Some embodiments provide for the identification of MRSA comprising type i, ii, iii, iv, v, vii, and xxi MREJ nucleic acids, using a combination of MREJ-specific oligonucleotides disclosed herein. Some embodiments provide for the detection of MRSA comprising type i, ii, iii, iv, v, vi, vii, ix, xiii, xiv, and xxi nucleic acids, using a combination of MREJ-specific oligonucleotides disclosed herein. Some embodiments provide for the identification of MRSA comprising type i, ii, iii, iv, v, vi, vii, ix, xiii, xiv, and xxi nucleic acids, using a combination of MREJ-specific oligonucleotides disclosed herein. Some embodiments provide for the detection and/or identification of MRSA comprising type i, ii, iii, iv, vii, xvi, and xxi nucleic acids, using a combination of MREJ-specific disclosed herein.

### Probes

In some embodiments, sequence-specific probes are provided. Probes include, but are not limited to oligonucleotides as described herein. In some embodiments, sequence-specific probes disclosed herein specifically hybridize to a target sequence, such as an MREJ type xxi region nucleic acid sequence. For example, in some embodiments, sequence specific probes disclosed herein specifically hybridize to SEQ ID NO:1, or the complement thereof, or a subsequence thereof (e.g., an amplicon of a region within SEQ ID NO:1). In some embodiments, the sequence-specific probe specifically hybridizes to, and is fully or substantially complementary a nucleotide sequence flanked by the binding sites of a forward primer and reverse primer disclosed herein. In some embodiments, the sequence specific probes comprise at least 5, 6, 7, 8, 9, 10, 11, 12, 13, 14, 15, 16, 17, 18, 19, 20, 21, 22, 23, 24, or 25 nucleotides of SEQ ID NO: 2 or 3, such that the sequence specific probe overlaps with the binding site of an amplification primer disclosed herein.

In some embodiments, sequence-specific probes that hybridize to orfX are provided. In some embodiments, sequence specific probes that hybridize to mecA are provided. In some embodiments, sequence specific probes that hybridize to mecC are provided. In some embodiments, sequence specific probes that hybridize to nuc are provided. In some embodiments, sequence specific probes that hybridize to femB are provided. In some embodiments, sequence specific probes that hybridize to Sa442 are provided.

The skilled artisan will readily appreciate that cognate pairs of amplification primers and sequence-specific probes can be provided together. That is, in embodiments where amplification primers that specifically hybridize to and generate an amplicon for a particular sequence are provided, embodiments disclosed herein can also include a sequence-specific probe that hybridizes to and is specific for the amplicon.

In some embodiments, the sequence specific probes disclosed herein specifically hybridize to the polymorphic right extremity sequences of an MREJ type xxi region under standard conditions for nucleic acid amplification, and/or stringent hybridization conditions. In some embodiments, the sequence specific probes disclosed herein specifically hybridize to the polymorphic right extremity sequences of an MREJ type i region under standard conditions for nucleic acid amplification, and/or stringent hybridization conditions. In some embodiments, the sequence specific probes disclosed herein specifically hybridize to the polymorphic right extremity sequences of an MREJ type ii region under standard conditions for nucleic acid amplification, and/or stringent hybridization conditions. In some embodiments, the sequence specific probes disclosed herein specifically hybridize to the polymorphic right extremity sequences of an MREJ type iii region under standard conditions for nucleic acid amplification, and/or stringent hybridization conditions. In some embodiments, the sequence specific probes disclosed herein specifically hybridize to the polymorphic right extremity sequences of an MREJ type iv region under standard conditions for nucleic acid amplification, and/or stringent hybridization conditions. In some embodiments, the sequence specific probes disclosed herein specifically hybridize to the polymorphic right extremity sequences of an MREJ type v region under standard conditions for nucleic acid amplification, and/or stringent hybridization conditions. In some embodiments, the sequence specific probes disclosed herein specifically hybridize to the polymorphic right extremity sequences of an MREJ type vi region under standard conditions for nucleic acid amplification, and/or stringent hybridization conditions. In some embodiments, the sequence specific probes disclosed herein specifically hybridize to the polymorphic right extremity sequences of an MREJ type vii region under standard conditions for nucleic acid amplification, and/or stringent hybridization conditions. In some embodiments, the sequence specific probes disclosed herein specifically hybridize to the polymorphic right extremity sequences of an MREJ type viii region under standard conditions for nucleic acid amplification, and/or stringent hybridization conditions. In some embodiments, the sequence specific probes disclosed herein specifically hybridize to the polymorphic right extremity sequences of an MREJ type ix region under standard conditions for nucleic acid amplification, and/or stringent hybridization conditions. In some embodiments, the sequence specific probes disclosed herein specifically hybridize to the polymorphic right extremity sequences of an MREJ type x region under standard conditions for nucleic acid amplification, and/or stringent hybridization conditions. In some embodiments, the sequence specific probes disclosed herein specifically hybridize to the polymorphic right extremity sequences of an MREJ type xi region under standard conditions for nucleic acid amplification, and/or stringent hybridization conditions. In some embodiments, the sequence specific probes disclosed herein specifically hybridize to the polymorphic right extremity sequences of an MREJ type xii region under standard conditions for nucleic acid amplification, and/or stringent hybridization conditions. In some embodiments, the sequence specific probes disclosed herein specifically hybridize to the polymorphic right extremity sequences of an MREJ type xiii region under standard conditions for nucleic acid amplification, and/or stringent hybridization conditions. In some embodiments, the sequence specific probes disclosed herein specifically hybridize to the polymorphic right extremity sequences of an MREJ type xiv region under standard conditions for nucleic acid amplification, and/or stringent hybridization conditions. In some embodiments, the sequence specific probes disclosed herein specifically hybridize to the polymorphic right extremity sequences of an MREJ type xv region under standard conditions for nucleic acid amplification, and/or stringent hybridization conditions. In some embodiments, the sequence specific probes disclosed herein specifically hybridize to the polymorphic right extremity sequences of an MREJ type xvi region under standard conditions for nucleic acid amplification, and/or stringent hybridization conditions. In some embodiments, the sequence specific probes disclosed herein specifically hybridize to the polymorphic right extremity sequences of an MREJ type xvii region under standard conditions for nucleic acid amplification, and/or stringent hybridization conditions. In some embodiments, the sequence specific probes disclosed herein specifically hybridize to the polymorphic right extremity sequences of an MREJ type xviii region under standard conditions for nucleic acid amplification, and/or stringent hybridization conditions. In some embodiments, the sequence specific probes disclosed herein specifically hybridize to the polymorphic right extremity sequences of an MREJ type xix region under standard conditions for nucleic acid amplification, and/or stringent hybridization conditions. In some embodiments, the sequence specific probes disclosed herein specifically hybridize to the polymorphic right extremity sequences of an MREJ type xx region under standard conditions for nucleic acid amplification, and/or stringent hybridization conditions. In some embodiments, the sequence specific probes disclosed herein specifically hybridize to mecA sequences under standard conditions for nucleic acid amplification, and/or stringent hybridization conditions. In some embodiments, the sequence specific probes disclosed herein specifically hybridize to mecC sequences under standard conditions for nucleic acid amplification, and/or stringent hybridization conditions. In some embodiments, the sequence specific probes disclosed herein specifically hybridize to nuc sequences under standard conditions for nucleic acid amplification, and/or stringent hybridization conditions. In some embodiments, the sequence specific probes disclosed herein specifically hybridize to femB sequences under standard conditions for nucleic acid amplification, and/or stringent hybridization conditions. In some embodiments, the sequence specific probes disclosed herein specifically hybridize to Sa442 sequences under standard conditions for nucleic acid amplification, and/or stringent hybridization conditions.

In some embodiments, the sequence specific probes disclosed herein specifically hybridize to S. aureus chromosomal sequences located within 1 kilobase from the insertion point of the SCCmec element into the chromosomal DNA. For example, in some embodiments, provided is a sequence specific probe that specifically hybridizes to S. aureus orfX sequences under standard conditions for nucleic acid amplification, and/or stringent hybridization conditions. In some embodiments, provided is a sequence specific probe that hybridizes to orfSA0022 under standard conditions for nucleic acid amplification. In some embodiments, the sequence specific probes disclosed herein specifically hybridize to the MREP sequences, e.g., MREP type i, ii, iii, iv, v, vi, vii, viii, ix, x, xi, xii, xiii, xiv, xv, xvi, xvii, xviii, xix, xx, or xxi sequences. In some embodiments, more than one sequence specific probe is provided. For example, in some embodiments, sequence specific probes that specifically hybridize to MREP type xxi sequences are provided in combination with one, two, three, four, five, six, seven, eight, nine, ten, eleven, twelve, thirteen, fourteen, fifteen, sixteen, seventeen, eighteen, nineteen or twenty, or more, sequence specific probes.

In some embodiments, oligonucleotide probes can include a detectable moiety. For example, in some embodiments, the oligonucleotide probes disclosed herein can comprise a radioactive label. Non-limiting examples of radioactive labels include ³H, ¹⁴C, ³²P, and ³⁵S. In some embodiments, oligonucleotide probes can include one or more non-radioactive detectable markers or moieties, including but not limited to ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe. For example, oligonucleotide probes labeled with one or more dyes, such that upon hybridization to a template nucleic acid, a detectable change in fluorescence is generated. While non-specific dyes may be desirable for some applications, sequence-specific probes can provide more accurate measurements of amplification. One configuration of sequence-specific probe can include one end of the probe tethered to a fluorophore, and the other end of the probe tethered to a quencher. When the probe is unhybridized, it can maintain a stem-loop configuration, in which the fluorophore is quenched by the quencher, thus preventing the fluorophore from fluorescing. When the probe is hybridized to a template nucleic sequence, it is linearized, distancing the fluorophore from the quencher, and thus permitting the fluorophore to fluoresce. Another configuration of sequence-specific probe can include a first probe tethered to a first fluorophore of a FRET pair, and a second probe tethered to a second fluorophore of a FRET pair. The first probe and second probe can be configured to hybridize to sequences of an amplicon that are within sufficient proximity to permit energy transfer by FRET when the first probe and second probe are hybridized to the same amplicon.

In some embodiments, the sequence specific probe comprises an oligonucleotide as disclosed herein conjugated to a fluorophore. In some embodiments, the probe is conjugated to two or more fluorophore. Examples of fluorophores include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G) (emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, and the like.

In some embodiments, the probe is conjugated to a quencher. A quencher can absorb electromagnetic radiation and dissipate it as heat, thus remaining dark. Example quenchers include Dabcyl, NFQ's, such as BHQ-1 or BHQ-2 (Biosearch), IOWA BLACK FQ (IDT), and IOWA BLACK RQ (IDT). In some embodiments, the quencher is selected to pair with a fluorphore so as to absorb electromagnetic radiation emitted by the fluorophore. Flourophore/quencher pairs useful in the compositions and methods disclosed herein are well-known in the art, and can be found, e.g., described in S. Marras, “Selection of Fluorophore and Quencher Pairs for Fluorescent Nucleic Acid Hybridization Probes” available at the world wide web site molecular-beacons.org/download/marras,mmb06%28335%293.pdf.

In some embodiments, a fluorophore is attached to a first end of the probe, and a quencher is attached to a second end of the probe. Attachment can include covalent bonding, and can optionally include at least one linker molecule positioned between the probe and the fluorophore or quencher. In some embodiments, a fluorophore is attached to a 5′ end of a probe, and a quencher is attached to a 3′ end of a probe. In some embodiments, a fluorophore is attached to a 3′ end of a probe, and a quencher is attached to a 5′ end of a probe. Examples of probes that can be used in quantitative nucleic acid amplification include molecular beacons, SCORPION™ probes (Sigma), TAQMAN™ probes (Life Technologies) and the like. Other nucleic acid detection technologies that are useful in the embodiments disclosed herein include, but are not limited to nanoparticle probe technology (See, Elghanian, et al. (1997) Science 277:1078-1081.) and Amplifluor probe technology (See, U.S. Pat. Nos. 5,866,366; 6,090,592; 6,117,635; and 6,117,986).

Some embodiments disclosed herein provide probes that specifically hybridize to an MREJ type xxi sequence, or an amplicon of an MREJ type xxi sequence, e.g., SEQ ID NO: 1, or a subsequence thereof. Accordingly, some embodiments disclosed herein provide a probe that hybridizes to an amplicon from the amplification of a template comprising SEQ ID NO: 1 using the amplification primers SEQ ID NOs: 2 and 3. By way of example only, in some embodiments, the probe can comprise, consist essentially of, or consist of the sequence of SEQ ID NO: 4, or a variant thereof, is provided. In some embodiments, the probe comprises a fluorophore and/or quencher as described herein. In some embodiments, the probe can comprise, consist essentially of, or consist of the sequence of SEQ ID NO: 82, or a variant thereof, is provided. In some embodiments, the probe comprises a fluorophore and/or quencher as described herein. In preferred embodiments, probes can comprise SEQ ID NO: 4 or SEQ ID NO:82, or variants thereof, with the fluorophore 6-carboxyfluorescein (“FAM”) attached to the 5′ end of the probe, and the quencher IOWA BLACK Black-hole Quencher® 2 (IDT) (“BHQ”) attached to the 3′ end of the probe.

### Kits

Also provided herein are kits. The kits, primers and probes disclosed herein can be used to detect and/or identify MRSA of MREJ type xxi (and, in some embodiments, additionally to detect and/or identify MRSA of one or more of MREJ types i-xx), in both in vitro and/or in situ applications. For example, it is contemplated that the kits may be used in combination with any previously described primers/probes for detecting MRSA of MREJ types i to xx. It is also contemplated that the diagnostic kits, primers and probes disclosed herein can be used alone or in combination with any other assay suitable to detect and/or identify microorganisms, including but not limited to: any assay based on nucleic acids detection, any immunoassay, any enzymatic assay, any biochemical assay, any lysotypic assay, any serological assay, any differential culture medium, any enrichment culture medium, any selective culture medium, any specific assay medium, any identification culture medium, any enumeration culture medium, any cellular stain, any culture on specific cell lines, and any infectivity assay on animals.

Accordingly, in some embodiments the kits disclosed herein will include an oligonucleotide that specifically binds to the polymorphic right extremity sequences of MREJ type xxi sequences (e.g., SEQ ID NO:1 or the complement thereof) under standard nucleic acid amplification conditions, and/or stringent hybridization conditions. For example, in some embodiments, the kits disclosed herein will include an oligonucleotide (e.g., a first amplification primer) that comprises, consists essentially of, or consists of SEQ ID NO:2, or a variant thereof. In some embodiments, the kit can also include one or more additional oligonucleotides, e.g., a second amplification primer such as an oligonucleotide that comprises, consists essentially of, or consists of SEQ ID NO:3, or a variant thereof, that, together with a first amplification primer will generate an amplicon of the polymorphic right extremity junction of MREJ type xxi sequences. In some embodiments, the kit can also include a probe as described herein. For example, in some embodiments, the kit can include an oligonucleotide probe that comprises, consists essentially of, or consists of SEQ ID NO:4, or a variant thereof.

In some embodiments, the kits disclosed herein can include, in addition to an oligonucleotide that specifically binds to the polymorphic right extremity sequences of MREJ type xxi sequences under standard amplification conditions, one or more oligonucleotides that specifically bind to one or more of the SCCmec polymorphic right extremity sequences (MREP) within MREJ type i, ii, iii, iv, v, vi, vii, viii, ix, x, xi, xii, xiii, xiv, xv, xvi, xvii, xviii, xix, or xx. In some embodiments, the disclosed herein can include, in addition to an oligonucleotide that specifically binds to the polymorphic right extremity sequences of MREJ type xxi sequences under standard amplification conditions, one or more oligonucleotides that specifically bind to mecA sequences. In some embodiments, the kits disclosed herein can include, in addition to an oligonucleotide that specifically binds to the polymorphic right extremity sequences of MREJ type xxi sequences under standard amplification conditions, one or more oligonucleotides that specifically bind to mecC sequences. In some embodiments, the kits disclosed herein can include, in addition to an oligonucleotide that specifically binds to the polymorphic right extremity sequences of MREJ type xxi sequences under standard amplification conditions, one or more oligonucleotides that specifically bind to nuc sequences. In some embodiments, the kits disclosed herein can include, in addition to an oligonucleotide that specifically binds to the polymorphic right extremity sequences of MREJ type xxi sequences under standard amplification conditions, one or more oligonucleotides that specifically bind to S. aureus-specific nucleotide sequences, e.g., femB sequences. In some embodiments, the kits disclosed herein can include, in addition to an oligonucleotide that specifically binds to the polymorphic right extremity sequences of MREJ type xxi sequences under standard amplification conditions, one or more oligonucleotides that specifically bind to Sa442 sequences.

In some embodiments, provided are kits containing the reagents and compositions to carry out the methods described herein. Such a kit can comprise a carrier being compartmentalized to receive in close confinement therein one or more containers, such as tubes or vials. One of the containers may contain at least one unlabeled or detectably labeled primer or probe disclosed herein. The primer or primers can be present in lyophilized form or in an appropriate buffer as necessary. One or more containers may contain one or more enzymes or reagents to be utilized in, for example, nucleic acid amplification reactions reactions. These enzymes may be present by themselves or in admixtures, in lyophilized form or in appropriate buffers. Exemplary enzymes useful in nucleic acid amplification reactions as disclosed herein include, but are not limited to, FASTSTART™ Taq DNA polymerase, APTATAQ™ DNA polymerase (Roche), KLENTAQ 1™ DNA polymerase (AB peptides Inc.), HOTGOLDSTAR™ DNA polymerase (Eurogentec), KAPATAQ™ HotStart DNA polymerase, KAPA2G™ Fast HotStart DNA polymerase (Kapa Biosystemss), PHUSION™ Hot Start DNA Polymerase (Finnzymes), or the like.

Additionally, the kits disclosed herein can include all of the additional elements necessary to carry out the methods disclosed herein, such as buffers, extraction reagents, enzymes, pipettes, plates, nucleic acids, nucleoside triphosphates, filter paper, gel materials, transfer materials, autoradiography supplies, and the like.

In some embodiments, the kits include additional reagents that are required for or convenient and/or desirable to include in the reaction mixture prepared during the methods disclosed herein, where such reagents include: one or more polymerases; an aqueous buffer medium (either prepared or present in its constituent components, where one or more of the components may be premixed or all of the components may be separate), and the like. The various reagent components of the kits may be present in separate containers, or may all be pre-combined into a reagent mixture for combination with template nucleic acid.

In addition to the above components, in some embodiments, the kits can also include instructions for practicing the methods disclosed herein. These instructions can be present in the kits in a variety of forms, one or more of which may be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address that may be used via the internet to access the information at a removed site. Any convenient means may be present in the kits.

### Methods

Provided herein are methods for the detection, identification and/or quantification of MRSA having MREJ type xxi nucleic acids from a sample. In some embodiments, the methods include the step of contacting the sample to be analyzed with an oligonucleotide that specifically hybridizes to the polymorphic right extremity sequences of an SCCmec MREJ type xxi region under standard nucleic acid amplification conditions and/or stringent hybridization conditions.

In some embodiments, a sample to be tested for the presence of an MRSA having an SCCmec MREJ type xxi region is processed prior to performing the methods disclosed herein. For example, in some embodiments, the sample can be isolated, concentrated, or subjected to various other processing steps prior to performing the methods disclosed herein. For example, in some embodiments, the sample can be processed to isolate nucleic acids from the sample prior to contacting the sample with the oligonucleotides, as disclosed herein. As used herein, the phrase “isolate nucleic acids” refers to the purification of nucleic acids from one or more cellular components. The skilled artisan will appreciate that samples processed to “isolate nucleic acids” therefrom can include components and impurities other than nucleic acids. In some embodiments, the methods disclosed herein are performed on the sample without culturing the sample in vitro. In some embodiments, the methods disclosed herein are performed on the sample without isolating nucleic acids from the sample prior to contacting the sample with oligonucleotides as disclosed herein.

i. Non-Amplification Based Methods

In some embodiments, the oligonucleotide comprises a detectable moiety, as described elsewhere herein, and the specific hybridization of the oligonucleotide to the polymorphic right extremity sequences of an SCCmec MREJ type xxi region can be detected, e.g., by direct or indirect means. Accordingly, some embodiments for the detection and/or identification of methicillin-resistant Staphylococcus aureus (MRSA) comprising MREJ type xxi nucleic acids, that include the steps of providing a test sample; and contacting the sample with an oligonucleotide probe that specifically hybridizes to the polymorphic right extremity sequences of an SCCmec MREJ type xxi region under standard nucleic acid amplification conditions and/or stringent hybridization conditions, wherein the oligonucleotide probe is between 10 and 45 nucleotides in length, and comprises a detectable moiety, wherein the contacting is performed under conditions allowing for the specific hybridization of the primer to the mec right extremity junction of the MREJ type xxi sequence if S. aureus comprising MREJ type xxi sequences is present in the sample. The presence and/or amount of probe that is specifically bound to mec right extremity junction of the MREJ type xxi sequence (if present in the sample being tested) can be determined, wherein bound probe is indicative of the presence of an MRSA having SCCmec MREJ type xxi sequences in the sample. In some embodiments, the amount of bound probe is used to determine the amount of MRSA having SCCmec MREJ type xxi sequences in the sample.

Similarly, in some embodiments, non-amplification based methods can be used to detect additional nucleotide sequences. For example, in some embodiments, the methods disclosed herein can include the use of non-amplification based methods to detect the presence and/or amount of other MREJ sequences, e.g., one or more of MREJ types i, ii, iii, iv, v, vi, vii, viii, ix, x, xi, xii, xiii, xiv, xv, xvi, xvii, xviii, xix, or xx, or any combination thereof, in addition to MREJ type xxi sequences. In some embodiments, the methods disclosed herein can include the use of non-amplification based methods to detect the presence of mecA sequences, in addition to MREJ (or MREP) type xxi sequences. In some embodiments, the methods disclosed herein can include the use of non-amplification based methods to detect the presence of mecC sequences, in addition to (MREP) MREJ type xxi sequences. In some embodiments, the methods disclosed herein can include the use of non-amplification based methods to detect the presence of S. aureus specific sequences, such as nuc sequences, femB sequences, Sa442 sequences, 16S rRNA sequences, or the like in addition to (MREP) MREJ type xxi sequences. Accordingly, in an exemplary embodiment, provided herein are methods and compositions for the simultaneous detection of MREJ type xxi, i, ii, iii, iv, vii, mecA, mecC, and nuc sequences.

The determining step can be achieved using any methods known to those skilled in the art, including but not limited to, in situ hybridization, following the contacting step. The detection of hybrid duplexes (i.e., of a probe specifically bound to polymorphic right extremity sequences from an MREJ type xxi region) can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample. Those skilled in the art will appreciate that wash steps may be employed to wash away excess sample/target nucleic acids or oligonucleotide probe (as well as unbound conjugate, where applicable). Further, standard heterogeneous assay formats are suitable for detecting the hybrids using the labels present on the oligonucleotide primers and probes.

Thus, according to some embodiments, the methods disclosed herein can include, for example ELISA (e.g., in a dipstick format, a multi-well format, or the like) using art-recognized methods.

ii. Amplification-Based Methods

In some embodiments, the methods for the detection and/or identification of methicillin-resistant Staphylococcus aureus (MRSA) comprising MREJ type xxi nucleic acids, that include the steps of providing a test sample; and contacting the sample with an oligonucleotide probe that specifically hybridizes to the polymorphic right extremity sequences of an SCCmec MREJ type xxi region under standard nucleic acid amplification conditions and/or stringent hybridization conditions, wherein the oligonucleotide probe is between 10 and 45 nucleotides in length.

In some embodiments, the sample is contacted under standard amplification conditions, or conditions allowing for the specific hybridization and extension of the primer to the mec right extremity polymorphic sequence (MREP sequence) of the MREJ type xxi sequence if S. aureus comprising MREJ type xxi sequences is present in the sample. Accordingly, the methods include the step of specific amplification of MREJ type xxi nucleic acids from samples, e.g., to generate amplicons or amplification products that include the mec right extremity junction of an MREJ type xxi sequence. In some embodiments, the sample is contacted under standard amplification conditions, or conditions allow for the specific hybridization of a primer pair, e.g., a first primer that hybridizes to the mec right extremity polymorphic sequence (MREP sequence) and a second primer that hybridizes to the S. aureus chromosomal sequence adjacent to the SCCmec right extremity, i.e., orfX in order to generate an amplicon across the SCCmec-chromosomal junction. Some embodiments provide methods to generate SCCmec right extremity junction sequence data by contacting a sample under standard amplification conditions, or conditions allow for the specific hybridization of a primer pair, e.g., a first primer that hybridizes to the mec right extremity polymorphic sequence (MREP sequence) and a second primer that hybridizes to the S. aureus chromosomal sequence adjacent to the SCCmec right extremity, i.e., orfX in order to generate an amplicon across the SCCmec-chromosomal junction.

In some embodiments, the sample is contacted, e.g., simultaneously with (as in multiplex PCR), or sequentially to the contacting with the MREJ type xxi-specific oligonucleotide(s), under the same standard amplification conditions, with additional primers that allow for the specific amplification of one or more additional MREJ type sequences, e.g., one or more of MREJ type i, ii, iii, iv, v, vi, vii, viii, ix, x, xi, xii, xiii, xiv, xv, xvi, xvii, xviii, xix, or xx sequences. In some embodiments, the sample is contacted, e.g., simultaneously with (multiplex PCR), or sequentially to, the contacting with MREJ type xxi-specific oligonucleotide(s), under the same standard amplification conditions, with additional primers that allow for the specific amplification of mecA and/or mecC sequences. In some embodiments, the sample is contacted, e.g., simultaneously with (multiplex PCR), or sequentially to, the contacting with MREJ type xxi-specific oligonucleotide(s), under the same standard amplification conditions, with additional primers that allow for the specific amplification of one or more sequences that is unique to S. aureus (S. aureus-specific sequences), such as nuc, femB, Sa442, and the like. Accordingly, in some embodiments, the sample is contacted with primers specific for MREP type xxi, i, ii, iii, iv, v, vii, ix, xiii, xiv, and xxi sequences, as well as mecA, mecC and nuc sequences.

In some embodiments, the methods include the identification of a specific type of sequence (e.g., an MREJ type xxi sequence). For example, in embodiments involving the specific amplification of only MREJ tye xxi sequences in simplexm the presence or the absence of an amplicon is indicative of the presence or absence of MREJ type xxi sequences in the sample. In some embodiments, the methods involve the specific amplification of additional sequences, e.g., MREJ sequences, mec sequences, S. aureus specific sequences, and the like, in multiplex with the specific amplification of MREJ type xxi sequences. In the embodiments that involve multiplex amplification, in some embodiments, the methods can include the identification or detection of specific sequences, e.g., by using sequence specific probes that hybridize to only one amplicon. In some embodiments, the methods do not discriminate between some or all of the different possible amplicons present in the sample after amplification. For example, in some embodiments, a sequence specific probe that hybridizes to orfX can be used to detect the presence of amplicons of one or more MREJ types. In some embodiments, the methods include the detection of an amplification product, without the specific detection of a particular sequence.

Several methods for the specific amplification of target nucleic acids are known in the art, and are useful in the embodiments disclosed herein. Non-limiting examples of amplification methods include Polymerase Chain Reaction (PCR; see Saiki et al., 1985, Science 230:1350-1354, herein incorporated by reference), Ligase Chain Reaction (LCR; see Wu et al., 1989, Genomics 4:560-569; Barringer et al., 1990, Gene 89:117-122; Barany, 1991, Proc. Natl. Acad. Sci. USA 88:189-193, all of which are incorporated herein by reference), Transcription Mediated Amplification (TMA; see Kwoh et al., 1989, Proc. Natl. Acad. Sci. USA 86:1173-1177, herein incorporated by reference), Self-Sustaining Sequence Replication (3SR; see Guatelli et al., 1990, Proc. Natl. Acad. Sci. USA 87:1874-1878, herein incorporated by reference), Rolling Circle Amplification (RCA), Nucleic Acid Sequence Based Amplification (NASBA), Q 13 replicase system (Lizardi et al., 1988, BioTechnology 6:1197-1202, herein incorporated by reference) and Strand Displacement Amplification (SDA; see Walker et al., 1992, Proc. Natl. Acad. Sci. USA 89:392-396; Walker et al., 1992, Nuc. Acids. Res. 20:1691-1696; and EP 0 497 272, all of which are incorporated herein by reference)) including thermophilic SDA (tSDA).

In various embodiments, the methods disclosed herein are useful for detecting the presence of SCCmec MREJ type xxi nucleic acids or sequences in clinical samples. For example, in some embodiments, the methods disclosed herein are useful for detecting and identifying S. aureus having type xxi MREJ regions samples having concentration of bacteria that is within physiological ranges (i.e., the concentration of bacteria in a sample collected from a subject infected with the bacteria). Thus, a sample can be directly screened without the need for isolating, concentrating, or expanding (e.g., culturing) the bacterial population in order to detect the presence of MRSA having MREJ type xxi nucleic acids. In various embodiments, the methods disclosed herein are capable of detecting the presence of a MRSA having MREJ type xxi nucleic acids from a sample that has a concentration of bacteria of about 1 CFU/ml, 10 CFU/ml, 100 CFU/ml, 1×10³ CFU/ml, 1×10³ CFU/ml, about 1×10⁴ CFU/ml, about 1×10⁵ CFU/ml, or about 1×10⁶ CFU/ml, or any number in between.

In some embodiments, the methods described herein, the methods include the performance of PCR or qPCR in order to generate an amplicon. Numerous different PCR and qPCR protocols are known in the art and exemplified herein below and can be directly applied or adapted for use using the presently described compositions for the detection and/or identification of MRSA having MREJ type xxi nucleic acids in a sample.

Generally, in PCR, a target polynucleotide sequence is amplified by reaction with at least one oligonucleotide primer or pair of oligonucleotide primers. The primer(s) specifically hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence that is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, SMARTCYCLER® (Cepheid, Sunnyvale, Calif.), ABI PRISM 7700® (Applied Biosystems, Foster City, Calif.), ROTOR-GENE™; (Corbett Research, Sydney, Australia), LIGHTCYCLER® (Roche Diagnostics Corp, Indianapolis, Ind.), ICYCLER® (Biorad Laboratories, Hercules, Calif.) and MX4000® (Stratagene, La Jolla, Calif.)

Some embodiments provide methods including Quantitative PCR (qPCR) (also referred as real-time PCR). qPCR can provide quantitative measurements, and also provide the benefits of reduced time and contamination. As used herein, “quantitative PCR” (or “real time qPCR”) refers to the direct monitoring of the progress of a PCR amplification as it is occurring without the need for repeated sampling of the reaction products. In qPCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence (herein referred to as cycle threshold or “CT”) varies directly with the concentration of amplifiable targets at the beginning of the PCR process, enabling a measure of signal intensity to provide a measure of the amount of target nucleic acid in a sample in real time.

Methods for setting up PCR and qPCR are well known to those skilled in the art. The reaction mixture minimally comprises template nucleic acid (e.g., as present in test samples, except in the case of a negative control as described below) and oligonucleotide primers and/or probes in combination with suitable buffers, salts, and the like, and an appropriate concentration of a nucleic acid polymerase. As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-direction along the template until synthesis terminates. An appropriate concentration includes one that catalyzes this reaction in the presently described methods. Known DNA polymerases useful in the methods disclosed herein include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase, FASTSTART™ Taq DNA polymerase, APTATAQ™ DNA polymerase (Roche), KLENTAQ 1™ DNA polymerase (AB peptides Inc.), HOTGOLDSTAR™ DNA polymerase (Eurogentec), KAPATAQ™ HotStart DNA polymerase, KAPA2G™ Fast HotStart DNA polymerase (Kapa Biosystemss), PHUSION™ Hot Start DNA Polymerase (Finnzymes), or the like.

In addition to the above components, the reaction mixture of the present methods includes primers, probes, and deoxyribonucleoside triphosphates (dNTPs).

Usually the reaction mixture will further comprise four different types of dNTPs corresponding to the four naturally occurring nucleoside bases, i.e., dATP, dTTP, dCTP, and dGTP. In the methods of the invention, each dNTP will typically be present in an amount ranging from about 10 to 5000 μM, usually from about 20 to 1000 μM, about 100 to 800 μM, or about 300 to 600 μM.

The reaction mixture can further include an aqueous buffer medium that includes a source of monovalent ions, a source of divalent cations, and a buffering agent. Any convenient source of monovalent ions, such as potassium chloride, potassium acetate, ammonium acetate, potassium glutamate, ammonium chloride, ammonium sulfate, and the like may be employed. The divalent cation may be magnesium, manganese, zinc, and the like, where the cation will typically be magnesium. Any convenient source of magnesium cation may be employed, including magnesium chloride, magnesium acetate, and the like. The amount of magnesium present in the buffer may range from 0.5 to 10 mM, and can range from about 1 to about 6 mM, or about 3 to about 5 mM. Representative buffering agents or salts that may be present in the buffer include Tris, Tricine, HEPES, MOPS, and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, for example, about pH 6.0, 6.5, 7.0, 7.5, 8.0, 8.5, 9.0, or 9.5. Other agents that may be present in the buffer medium include chelating agents, such as EDTA, EGTA, and the like. In some embodiments, the reaction mixture can include BSA, or the like. In addition, in some embodiments, the reactions can include a cryoprotectant, such as trehalose, particularly when the reagents are provided as a master mix, which can be stored over time.

In preparing a reaction mixture, the various constituent components may be combined in any convenient order. For example, the buffer may be combined with primer, polymerase, and then template nucleic acid, or all of the various constituent components may be combined at the same time to produce the reaction mixture.

Alternatively, commercially available premixed reagents can be utilized in the methods disclosed herein, according to the manufacturer's instructions, or modified to improve reaction conditions (e.g., modification of buffer concentration, cation concentration, or dNTP concentration, as necessary), including, for example, TAQMAN® Universal PCR Master Mix (Applied Biosystems), OMNIMIX® or SMARTMIX® (Cepheid), IQ&#8482; Supermix (Bio-Rad Laboratories), LIGHTCYCLER® FastStart (Roche Applied Science, Indianapolis, Ind.), or BRILLIANT® QPCR Master Mix (Stratagene, La Jolla, Calif.).

The reaction mixture can be subjected to primer extension reaction conditions (“conditions sufficient to provide polymerase-based nucleic acid amplification products”), i.e., conditions that permit for polymerase-mediated primer extension by addition of nucleotides to the end of the primer molecule using the template strand as a template. In many embodiments, the primer extension reaction conditions are amplification conditions, which conditions include a plurality of reaction cycles, where each reaction cycle comprises: (1) a denaturation step, (2) an annealing step, and (3) a polymerization step. As discussed below, in some embodiments, the amplification protocol does not include a specific time dedicated to annealing, and instead comprises only specific times dedicated to denaturation and extension. The number of reaction cycles will vary depending on the application being performed, but will usually be at least 15, more usually at least 20, and may be as high as 60 or higher, where the number of different cycles will typically range from about 20 to 40. For methods where more than about 25, usually more than about 30 cycles are performed, it may be convenient or desirable to introduce additional polymerase into the reaction mixture such that conditions suitable for enzymatic primer extension are maintained.

The denaturation step comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture will usually be raised to, and maintained at, a temperature ranging from about 85 to 100° C., usually from about 90 to 98° C., and more usually from about 93 to 96° C., for a period of time ranging from about 3 to 120 sec, usually from about 3 sec.

Following denaturation, the reaction mixture can be subjected to conditions sufficient for primer annealing to template nucleic acid present in the mixture (if present), and for polymerization of nucleotides to the primer ends in a manner such that the primer is extended in a 5′ to 3′ direction using the nucleic acid to which it is hybridized as a template, i.e., conditions sufficient for enzymatic production of primer extension product. In some embodiments, the annealing and extension processes occur in the same step. The temperature to which the reaction mixture is lowered to achieve these conditions will usually be chosen to provide optimal efficiency and specificity, and will generally range from about 50 to 85° C., usually from about 55 to 70° C., and more usually from about 60 to 68° C. In some embodiments, the annealing conditions can be maintained for a period of time ranging from about 15 sec to 30 min, usually from about 20 sec to 5 min, or about 30 sec to 1 minute, or about 30 seconds.

This step can optionally comprise one of each of an annealing step and an extension step with variation and optimization of the temperature and length of time for each step. In a two-step annealing and extension, the annealing step is allowed to proceed as above. Following annealing of primer to template nucleic acid, the reaction mixture will be further subjected to conditions sufficient to provide for polymerization of nucleotides to the primer ends as above. To achieve polymerization conditions, the temperature of the reaction mixture will typically be raised to or maintained at a temperature ranging from about 65 to 75° C., usually from about 67 to 73° C. and maintained for a period of time ranging from about 15 sec to 20 min, usually from about 30 sec to 5 min. In some embodiments, the methods disclosed herein do not include a separate annealing and extension step. Rather, the methods include denaturation and extension steps, without any step dedicated specifically to annealing.

The above cycles of denaturation, annealing, and extension may be performed using an automated device, typically known as a thermal cycler. Thermal cyclers that may be employed are described elsewhere herein as well as in U.S. Pat. Nos. 5,612,473; 5,602,756; 5,538,871; and 5,475,610; the disclosures of which are herein incorporated by reference.

The methods described herein can also be used in non-PCR based applications to detect a target nucleic acid sequence, where such target may be immobilized on a solid support. Methods of immobilizing a nucleic acid sequence on a solid support are known in the art and are described in Ausubel et ah, eds. (1995) Current Protocols in Molecular Biology (Greene Publishing and Wiley-Interscience, NY), and in protocols provided by the manufacturers, e.g., for membranes: Pall Corporation, Schleicher &amp; Schuell; for magnetic beads: Dynal; for culture plates: Costar, Nalgenunc; for bead array platforms: Luminex and Becton Dickinson; and, for other supports useful according to the embodiments provided herein, CPG, Inc.

Variations on the exact amounts of the various reagents and on the conditions for the PCR or other suitable amplification procedure (e.g., buffer conditions, cycling times, etc.) that lead to similar amplification or detection/quantification results are known to those of skill in the art and are considered to be equivalents. In one embodiment, the subject qPCR detection has a sensitivity of detecting fewer than 50 copies (preferably fewer than 25 copies, more preferably fewer than 15 copies, still more preferably fewer than 10 copies, e.g. 5, 4, 3, 2, or 1 copy) of target nucleic acid (i.e., MREJ type xxi nucleic acids) in a sample.

### Controls

The assays disclosed herein can optionally include controls. PCR or qPCR reactions disclosed herein may contain various controls. Such controls can include a “no template” negative control, in which primers, buffer, enzyme(s) and other necessary reagents (e.g., MgCl₂, nucleotides, and the like) are cycled in the absence of added test sample. This ensures that the reagents are not contaminated with polynucleotides that are reactive with the primers, and that produce spurious amplification products. In addition to “no template” controls, negative controls can also include amplification reactions with non-specific target nucleic acid included in the reaction, or can be samples prepared using any or all steps of the sample preparation (from nucleic acid extraction to amplification preparation) without the addition of a test sample (e.g., each step uses either no test sample or a sample known to be free of carbapenem-resistant microorganisms).

In some embodiments, the methods disclosed herein can include a positive control, e.g., to ensure that the methods and reagents are performing as expected. The positive control can include known target that is unrelated to the MREJ type xxi target nucleic acids disclosed herein. Prior to amplification, the positive control nucleic acid (e.g., in the form of a plasmid that is either linearized or non-linearized) can be added to the amplification reaction. A single reaction may contain either a positive control template, a negative control, or a sample template, or a single reaction may contain both a sample template and a positive control. Preferably, the positive control will comprise sequences that are substantially complementary to the MREJ type xxi forward and reverse amplification primers derived from the MREJ type xxi sequences disclosed herein, such that an amplification primer pair used to amplify MREJ type xxi sequences will also amplify control nucleic acids under the same assay conditions. In some embodiments, the amplicon generated from the positive control template nucleic acids is larger than the target amplicon. Preferably, aside from the sequences in a positive control nucleic acid that are complementary or substantially complementary to the forward and reverse primers, the positive control nucleic acid will not share substantial similarity with the target amplicon/MREJ type xxi sequences disclosed herein. In other words, outside from the forward and reverse primers, the positive control amplicon is preferably less than 80%, less than 70%, less than 60%, less than 50%, less that 40%, less than 30%, less than 20%, and even more preferably, less than 10% identical with the positive control polynucleotide, e.g., when the sequence identity is compared using NCBI BLAST ALIGN tools.

Positive and/or negative controls can be used in setting the parameters within which a test sample will be classified as having or not having an MRSA having MREJ type xxi sequences. For example, in a qPCR reaction, the cycle threshold at which an amplicon is detected in a positive control sample can be used to set the threshold for classifying a sample as “positive,” and the cycle threshold at which an amplicon is detected in a negative control sample can be used to set the threshold for classifying a sample as “negative.” The CT from a single reaction may be used for each control, or the median or mean of replicate samples may be used. In yet another embodiment, historical control values may be used. The minimum level of detection for each of the negative and the positive controls is typically set at the lower end of the 95% confidence interval of the mean CT across multiple reactions. This value can be adjusted depending on the requirements of the diagnostic assay.

Preferably, PCR controls should be performed at the same time as the test sample, using the same reagents, in the same amplification reaction.

Some embodiments provide for the determination of the identity and/or amount of target amplification products, during the amplification reaction, e.g., in real-time. For example, some embodiments relate to taking measurements of, for example, probe that is specifically bound to target amplicon nucleic acids, and/or positive control amplicons (e.g., as indicated by fluorescence). In some embodiments, rather than using sequence-specific oligonucleotide probes, the methods can utilize non-sequence specific probes, which bind non-specifically to double-stranded nucleic acid, e.g., intercalating agents or the like. Intercalating agents have a relatively low fluorescence when unbound, and a relatively high fluorescence upon binding to double-stranded nucleic acids. As such, intercalating agents can be used to monitor the accumulation of double strained nucleic acids during a nucleic acid amplification reaction. Examples of such non-specific dyes include intercalating agents such as SYBR Green I™ (Molecular Probes), propidium iodide, ethidium bromide, LC green, SYTO9, EVAGREEN® fluorescent dye, CHROMOFY®, BEBO, and the like, that fluoresces and produces a detectable signal in the presence of double stranded nucleic acids. Measurements may be taken at a specified point during each cycle of an amplification reaction, e.g., after each extension step (prior to each denaturation step). Regardless of whether a sequence specific oligonucleotide probe or a non-sequence specific oligonucleotide probe is used, measurements of the amount of probe that is specifically bound to target amplicon nucleic acids, and/or positive control amplicons can be taken continuously throughout each cycle.

Alternatively, in some embodiments, the identity/amount of the amplicons (e.g., target and/or positive control) can be confirmed after the amplification reaction is completed, using standard molecular techniques including (for example) Southern blotting, dot blotting and the like.

### EXAMPLES

The following examples are provided to demonstrate particular situations and settings in which this technology may be applied and are not intended to restrict the scope of the invention and the claims included in this disclosure.

### Example 1

### Detection and Identification of MREJ Type XXI MRSA from Clinical Samples

A qPCR reaction to detect and identify MRSA having MREJ type xxi nucleic acids is performed. Clinical samples are collected from patients using Amies liquid swabs (Copan Diagnostics, Inc).

DNA is optionally isolated from the clinical samples using the BD GeneOhm™ Lysis kit (Becton Dickinson) pursuant to manufacturer's instructions. A sample of the isolated DNA is contacted with primers that specifically hybridize under standard amplification conditions to S. aureus species-specific orfX sequences and to the polymorphic right extremity sequences of MREJ type xxi, i.e., 0.2-0.7 μM each of SEQ ID NOs: 2, and 3. 0.3 μM dNTPs (Roche), 4 mM MgCl₂ (SIGMA), 2.8 units FASTSTART® Taq polymerase (Roche), 100 mM Tris, pH 8.3 (EMD), 10 mM KCl (LaboratoireMat), 5 mM (NH₄)₂SO₄ (SIGMA), 0.15 mg/mL BSA (SIGMA) 4% trehalose (SIGMA). The reaction also includes molecular beacon probes that specifically hybridize to amplification products of the right extremity junction of MREJ type xxi detectable, and which include detectable moieties detectable on the BD MAX™ (Becton Dickinson), SMARTCYCLER® (Cepheid) apparatus, or other apparatus configured for real-time PCR at FAM, Texas Red and Tet channels are added to the reaction mixture.

PCR is carried out in a BD MAX™ (Becton Dickinson) or SMARTCYCLER® (Cepheid) using the same cycling parameters as follows:

| | | Temp | | |
|---|---|---|---|---|
| Stage | Status | (° C.) | Sec | Optics |
| 1 | Hold | 95 | 900 | off |
| 2 | Repeat 45 | 95 | 1-5 | off |
| | times | 56-58 | 9, 10, or 15 | on |
| | | 72 | 10-20 | off |

The cycle threshold (CT) in FAM, Texas-Red, and TET channels is determined using the BD MAX™ or SMARTCYCLER® software.

### Example 2

### Multiplex Detection of MREJ Types I-XXI MRSA from Clinical Samples

A multiplex amplification reaction is performed to detect the presence of MRSA having any of MREJ types i-vii, ix, xiii, xiv and xxi is performed. Clinical samples are collected from patients using Amies liquid swabs (Copan Diagnostics, Inc).

DNA is optionally isolated from the clinical samples using the BD GeneOhm™ Lysis kit (Becton Dickinson) pursuant to manufacturer's instructions. A sample of the isolated DNA is contacted with primers that specifically hybridize under standard amplification conditions to S. aureus species-specific orfX sequences and to the polymorphic right extremity sequences of MREJ type i-vii, ix, xiii, xiv and xxi and i.e., 0.2-0.7 μM each of SEQ ID NOs: 2, 3, 39, 77, and 81, 0.3 μM dNTPs (Roche), 4 mM MgCl₂ (SIGMA), 2.8 units FASTSTART® Taq polymerase (Roche), 100 mM Tris, pH 8.3 (EMD), 10 mM KCl (LaboratoireMat), 5 mM (NH₄)₂SO₄ (SIGMA), 0.15 mg/mL BSA (SIGMA) 4% trehalose (SIGMA). The reaction also includes molecular beacon probes that specifically hybridize to amplification products of the right extremity junction of MREJ type xxi detectable, and which include detectable moieties detectable on the BD MAX™ (Becton Dickinson), SMARTCYCLER® (Cepheid) apparatus, or other apparatus configured for real-time PCR at FAM, Texas Red and Tet channels are added to the reaction mixture, i.e., SEQ ID NO:4.

PCR is carried out in a BD MAX™ (Becton Dickinson) or SMARTCYCLER® (Cepheid) using the same cycling parameters as follows:

| | | Temp | | |
|---|---|---|---|---|
| Stage | Status | (° C.) | Sec | Optics |
| 1 | Hold | 95 | 900 | off |
| 2 | Repeat 45 | 95 | 1-5 | off |
| | times | 56-58 | 9, 10, or 15 | on |
| | | 72 | 10-20 | off |

The cycle threshold (CT) in FAM, Texas-Red, and TET channels is determined using the BD MAX™ or SMARTCYCLER® software. The CT is used to determine whether MRSA of any of MREJ types i-vii, xvi, ix, xiii, xiv and xxi are present.

### Example 3

### MREJ Type XXI Sequences are Associated with the mecA Homolog, mecC

PCR amplification of the MREJ type xxi region and the mecC gene was performed on 51 isolates of MRSA isolated from either bovine or human hosts.

A list of the isolates is provided in the table below:

| | MREJ type | | | |
|---|---|---|---|---|
| | (confirmed by | | | MREJ |
| Strain | sequencing) | Host | Location | type |
| IDI6121 | xxi | Bovine | Somerset, England | xxi |
| IDI6122 | xxi | Bovine | Somerset, England | xxi |
| IDI6123 | xxi | Bovine | Bury St Edmunds, | xxi |
| | | | England | |
| IDI6124 | Unknown | Bovine | Langford, England | Unknown |
| IDI6125 | xxi | Bovine | Bury St Edmunds, | xxi |
| | | | England | |
| IDI6126 | xxi | Bovine | Sutton Bonington, | xxi |
| | | | England | |
| IDI6127 | xxi | Bovine | Sutton Bonington, | xxi |
| | | | England | |
| IDI6128 | xxi | Bovine | Sutton Bonington, | xxi |
| | | | England | |
| IDI6129 | xxi | Bovine | Sutton Bonington, | xxi |
| | | | England | |
| IDI6130 | xxi | Bovine | Sutton Bonington, | xxi |
| | | | England | |
| IDI6131 | xxi | Bovine | Sutton Bonington, | xxi |
| | | | England | |
| IDI6132 | xxi | Bovine | Sutton Bonington, | xxi |
| | | | England | |
| IDI6133 | xxi | Bovine | Sutton Bonington, | xxi |
| | | | England | |
| IDI6134 | xxi | Bovine | Thirsk, England | xxi |
| IDI6135 | xxi | Human | Tayside, Scotland | xxi |
| IDI6136 | xxi | Human | Lothian, Scotland | xxi |
| IDI6137 | xxi | Human | Scotland | xxi |
| IDI6138 | xxi | Human | Scotland | xxi |
| IDI6139 | xxi | Human | Scotland | xxi |
| IDI6140 | xxi | Human | Scotland | xxi |
| IDI6141 | xxi | Human | Scotland | xxi |
| IDI6142 | xxi | Human | Scotland | xxi |
| IDI6143 | xxi | Human | Scotland | xxi |
| IDI6144 | xxi | Human | Scotland | xxi |
| IDI6145 | xxi | Human | Scotland | xxi |
| IDI6146 | xxi | Human | Scotland | xxi |
| IDI6147 | xxi | Human | Denmark | xxi |
| IDI6148 | xxi | Human | Denmark | xxi |
| IDI6149 | xxi | Human | Denmark | xxi |
| IDI6150 | xxi | Human | Denmark | xxi |
| IDI6151 | xxi | Human | Denmark | xxi |
| IDI6152 | xxi | Human | Denmark | xxi |
| IDI6153 | xxi | Human | Denmark | xxi |
| IDI6154 | xxi | Human | Denmark | xxi |
| IDI6155 | xxi | Human | Denmark | xxi |
| IDI6156 | xxi | Human | Denmark | xxi |
| IDI6157 | xxi | Human | Denmark | xxi |
| IDI6158 | xxi | Human | Denmark | xxi |
| IDI6159 | xxi | Human | Denmark | xxi |
| IDI6160 | xxi | Human | Denmark | xxi |
| IDI6161 | xxi | Human | Denmark | xxi |
| IDI6162 | xxi | Human | Denmark | xxi |
| IDI6163 | xxi | Human | Denmark | xxi |
| IDI6164 | xxi | Human | Denmark | xxi |
| IDI6165 | xxi | Human | Denmark | xxi |
| IDI6166 | xxi | Human | Denmark | xxi |
| IDI6167 | xxi | Human | Denmark | xxi |
| IDI6168 | xxi | Human | France, Aix en | xxi |
| | | | provence | |
| IDI6170 | xxi | Human | France, Limoges | xxi |
| IDI6171 | xxi | Human | France, Aix en | xxi |
| | | | provence | |

DNA was isolated from the clinical samples above known to harbor mecC using the BD GeneOhm™ Lysis kit (Becton Dickinson) pursuant to manufacturer's instructions. The PCR reactions were prepared as follows: 0.2-0.7 μM each of SEQ ID NOs: 182, 183 and 187 0.3 μM dNTPs (Roche), 4 mM MgCl₂ (SIGMA), 2.8 units FASTSTART® Taq polymerase (Roche), 100 mM Tris, pH 8.3 (EMD), 10 mM KCl (LaboratoireMat), 5 mM (NH₄)₂SO₄ (SIGMA), 0.15 mg/mL BSA (SIGMA) 4% trehalose (SIGMA).

Reactions were performed on the BD MAX® system, using the FAM channel.

The data indicate that 50 of 51 MRSA strains that harbored the mecC gene contained an MREJ type xxi sequence, thus providing evidence of high ubiquity of detection of mecC MRSA strains using the MREJ type xxi sequence.

Having generally described this invention, a further understanding can be obtained by reference to certain specific examples which are provided herein for purposes of illustration only, and are not intended to be limiting.

What is claimed is:

1. A composition for the detection of methicillin-resistant Staphylococcus aureus (MRSA) comprising MREJ type xxi nucleic acids, said S. aureus comprising a Staphylococcal cassette chromosome mec (SCCmec) element including a mecA homolog (mecA_{LGA251}), said SCCmec cassette being inserted into S. aureus chromosomal DNA, thereby generating a polymorphic right extremity junction (MREJ) type xxi sequence that comprises polymorphic sequences from the right extremity and chromosomal DNA adjoining the polymorphic right extremity, said composition comprising:
a first amplification primer, said first amplification primer between 10 and 45 nucleotides in length, wherein said first amplification primer specifically hybridizes under standard PCR conditions to the polymorphic right extremity sequences of the MREJ type xxi nucleic acids.

2. The composition of claim 1, wherein the first amplification primer specifically hybridizes to the nucleic acid sequence of SEQ ID NO:1 or the complement thereof under said standard PCR conditions.

3. The composition of claim 1, further comprising:
a second amplification primer, said second amplification primer between 10 and 45 nucleotides in length, wherein said second amplification primer specifically hybridizes under standard PCR conditions to S. aureus chromosomal sequences located within 1 kilobase from the insertion point of the SCCmec element into the chromosomal DNA, and wherein said first and second amplification primer together generate an amplicon of the right extremity junction of MREJ type xxi nucleic acids under the standard PCR conditions in the presence of MRSA comprising MREJ type xxi nucleic acids.

4. The composition of claim 3, wherein said second amplification primer specifically hybridizes under standard PCR conditions to orfX.

5. The composition of claim 3, further comprising a probe, wherein said probe specifically hybridizes to the amplicon of the MREJ type xxi nucleic acids under the standard PCR conditions.

6. The composition of claim 1, further comprising a primer pair that specifically hybridizes within an is capable of amplification of a mecA sequence within SEQ ID NO:156.

7. The composition of claim 1, further comprising a primer pair that specifically hybridizes within and is capable of amplification of a mecC sequence within SEQ ID NO: 157.

8. The composition of claim 1, further comprising a primer pair that specifically hybridizes within and is capable of amplification of a nuc sequence within SEQ ID NO: 158.

9. The composition of claim 1, further comprising one or more additional amplification primers, wherein said one or more additional amplification primers are between 10 and 45 nucleotides in length, and wherein said one or more additional amplification primers specifically hybridize to one or more polymorphic SCCmec right extremity sequences from an MREJ type i to xx MRSA.

10. The composition of claim 9, wherein said one or more polymorphic SCCmec right extremity sequence is selected from the group consisting of SEQ ID NOs: 5 to 29.

11. The composition of claim 1, wherein the first amplification primer is at least 80% identical to SEQ ID NO:2.

12. The composition of claim 11, wherein the second amplification primer is at least 80% identical to SEQ ID NO:3.

13. The composition of claim 12, further comprising a probe, wherein the probe is at least 80% identical to SEQ ID NO:4 or 82.

14. The composition of claim 9, wherein said probe comprises a fluorescence emitter moiety and a fluorescence quencher moiety.

15. The composition of claim 1, wherein the first amplification primer is in lyophilized form.

16. A method for the detection of methicillin-resistant Staphylococcus aureus (MRSA) comprising MREJ type xxi nucleic acids, said S. aureus comprising a Staphylococcal cassette chromosome mec (SCCmec) element including a mecA homolog (mecA_{LGA251}), said SCCmec cassette being inserted into S. aureus chromosomal DNA, thereby generating a polymorphic right extremity junction (MREJ) type xxi sequence that comprises polymorphic sequences from the right extremity and chromosomal DNA adjoining the polymorphic right extremity, said method comprising:
providing a test sample;
contacting the sample with a first amplification primer, said first amplification primer between 10 and 45 nucleotides in length, wherein said first amplification primer specifically hybridizes under standard PCR conditions to the polymorphic right extremity sequences of the MREJ type xxi sequence;
contacting the sample with a second amplification primer between 10 and 45 nucleotides in length, wherein said second amplification primer hybridizes under the standard PCR conditions to the orfX gene of S. aureus, and wherein said first and second amplification primer together generate an amplicon of the SCCmec right extremity junction (MREJ) region sequence of the SCCmec right extremity junction of MRSA under the standard PCR conditions in the presence MREJ type xxi nucleic acids; and
determining whether or not an amplicon of the MREJ type xxi nucleic acids is generated following the contacting step.

17. The method of claim 16, wherein the first amplification primer specifically hybridizes to the MREP region of SEQ ID NO:1 under said standard PCR conditions.

18. The method of claim 16, wherein said contacting step further comprises contacting the sample with one or more additional amplification primers, wherein said one or more additional amplification primers are between 10 and 45 nucleotides in length, and wherein said one or more additional amplification primers specifically hybridizes to one or polymorphic SCCmec right extremity sequence from an MREJ type i to xx MRSA.

19. The method of claim 16, wherein said contacting step further comprises contacting the sample with one or more additional amplification primer pairs, wherein said one or more additional amplification primer pairs specifically hybridizes to, and is capable of generating an amplicon of, nuc sequences under the standard PCR conditions.

20. The method of claim 16, wherein said contacting step comprises performing multiplex PCR.

21. The method of claim 16, wherein said contacting step comprises performing real-time PCR.

22. The method of claim 16, wherein said contacting step further comprises contacting the sample with one or more additional amplification primer pairs, wherein said one or more additional amplification primer pairs specifically hybridizes to, and is capable of generating an amplicon of, mecA and/or mecC sequences under the standard PCR conditions.
